(19) United States
(12) Patent Application Publication
(10) Pub. No.: US 2025/0320241 A1
(43) Pub. Date: Oct. 16, 2025

(54) OXYGEN SENSING MATERIALS AND METHODS OF USE

(71) Applicants: David Jonathan Conway, West Palm Beach, FL (US); Kuntal Manna, New Delhi (IN); Chhaya Thadhani, New Delhi (IN); Manav Chauhan, Punjab (IN); Akanksha Choudhary, Uttar Pradesh (IN); Ramya Ahuja, Haryana (IN)

(72) Inventors: David Jonathan Conway, West Palm Beach, FL (US); Kuntal Manna, New Delhi (IN); Chhaya Thadhani, New Delhi (IN); Manav Chauhan, Punjab (IN); Akanksha Choudhary, Uttar Pradesh (IN); Ramya Ahuja, Haryana (IN)

(21) Appl. No.: 18/634,912

(22) Filed: Apr. 13, 2024

(51) Int. Cl. C07F 9/6558; A61B 5/00; A61K 49/00

(52) U.S. Cl. CPC C07F 9/65583; A61B 5/0082; A61K 49/0015; A61K 49/0052; A61B 5/0071

(57) ABSTRACT

Aspects of the disclosure generally relates to articles, compositions, and systems for oxygen detection in vitro or in vivo. In some embodiments, the disclosure relates to a metal complex, e.g., a compound comprising at least one metal-carbon bond. In some embodiments, the disclosure relates to metal organic frameworks (MOF). The MOFs comprise a plurality of metal clusters and a plurality of ligands that are coordinated with the plurality of metal clusters. In some embodiments, the disclosure relates to liposome-complexes (e.g., liposome-metal complexes and/or liposome-MOF complexes). In some embodiments, the metal complex, MOF, and/or liposome-complexes are configured to undergo photoluminescence following activation. Methods for in vitro and in vivo oxygen sensing and/or detecting intratumoral oxygen tensions using said articles, compositions, and system are also disclosed herein.

### FIELD

The present disclosure generally relates to metal complexes and metal organic frameworks for triggerable drug release using oxygen sensing.

### BACKGROUND

Tissue hypoxia refers to decreased oxygen levels relative to normal physiology and is associated with a plurality of pathologies, including, for example, solid tumors and non-healing ulcers. Currently, there are no delivery systems that enable drug delivery in response to low oxygen tissue levels. Thus, improvements are needed.

### SUMMARY

The subject matter of the present disclosure involves, in some cases, interrelated products, alternative solutions to a particular problem, and/or a plurality of different uses of one or more systems and/or articles.

Some aspects of the disclosure relate to complexes of Formula (I):

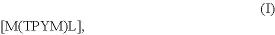

or a salt thereof, wherein:
M is Cu(I), Ir, Rh, Ag, Co, Fe, Ru, Ni, Zn, or Au; TPYM is Tris(2-pyridyl)methane, and L is a monophosphine ligand.

In some embodiments, the disclosure relates to compositions comprising a complex comprising a transition metal, TPYM, and a monophosphine ligand. In some embodiments, the complexes comprise a structure recited in Formula I

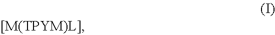

or a salt thereof, wherein M is the transition metal, wherein M is Cu(I), Ir, Rh, Ag, or Au; and L is the monophosphine ligand.

In some embodiments, the disclosure relates to one or more methods for using the metal complexes disclosed herein. For example, in some embodiments, the methods are directed toward in vitro oxygen-sensing. In some embodiments, the methods comprise adding a complex to an aqueous solution, wherein the complex comprises a transition metal, a TPYM group, and a monophosphine ligand; and determining photoluminescence intensity of the complex at a wavelength of between 440 nm to 480 nm.

In some embodiments, the methods relate to detecting intratumoral oxygen tension in a subject having, or suspected of having, a solid tumor. In some embodiments, the methods comprise administering a complex to the subject, wherein the complex comprises a transition metal, a TPYM group, and a monophosphine ligand. In some embodiments, the methods further comprise non-invasively monitoring a decrease in a photoluminescence intensity of the complex within the solid tumor, relative to the photoluminescence intensity of the complex in blood. In some embodiments, the methods comprise using a difference in photoluminescence intensity to determine the intratumoral oxygen tension in the subject.

Other aspects of the disclosure relate to metal organic frameworks (MOFs). In some embodiments, the MOFs comprise a plurality of metal clusters, wherein at least one metal cluster comprises a metal ion; and a plurality of ligands coordinating with the plurality of metal clusters, wherein the plurality of ligands comprises a triphenyldicarboxylic acid (TPDC) group or a triphenylphosphine group.

In some embodiments, the disclosure further relates to compositions comprising MOFs. For example, in some embodiments, the compositions comprise a metal organic framework (MOF) comprising a plurality of metal clusters and a plurality of ligands coordinating with the plurality of metal clusters, wherein at least one metal cluster comprises a metal ion, and wherein the plurality of ligands comprises a triphenyldicarboxylic acid (TPDC) derivative. In some embodiments, the compositions further comprise a liposome. In some embodiments, the compositions further comprise a cargo, e.g., a drug. In some embodiments, the compositions further comprise a pharmaceutically acceptable excipient.

In some embodiments, the disclosure relates to one or more methods for using the MOFs disclosed herein. For example, in some embodiments, the methods relate to in vitro oxygen sensing. In some embodiments, the methods comprise adding a metal organic framework to an aqueous solution, wherein the metal organic framework comprises a plurality of metal clusters and a plurality of ligands coordinating with the plurality of metal clusters, wherein at least one metal cluster comprises a metal ion, and wherein the plurality of ligands comprises a triphenyldicarboxylic acid (TPDC) group or a triphenylphosphine group. In some embodiments, the methods comprise monitoring a photoluminescence intensity of the metal organic framework at a wavelength of about 400 nm to 500 nm.

In some embodiments, the methods relate to detecting an intratumoral oxygen tension in a subject having, or suspected of having, a solid tumor. In some embodiments, the methods comprise administering a metal organic framework to the subject, wherein the metal organic framework comprises a plurality of metal clusters and a plurality of ligands coordinating with the plurality of metal clusters, wherein at least one metal cluster comprises a metal ion, and wherein the plurality of ligands comprises a triphenyldicarboxylic acid (TPDC) group or triphenylphosphine group. In some embodiments, the methods further comprise non-invasively monitoring a decrease in a photoluminescence intensity of the metal organic framework within the solid tumor, relative to the photoluminescence intensity of the metal organic framework in blood. Additionally, in some embodiments, the methods comprise using a difference in photoluminescence intensity to determine the intratumoral oxygen tension in the subject.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, metal complexes and/or MOFs. In still another aspect, the present disclosure encompasses methods of using one or more of the embodiments described herein, for example, metal complexes and/or MOFs.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a GC-MSD analysis of Tris(2-pyridyl)methane, according to some embodiments;

FIG. 2 shows a NMR of the prepared tri(o-tolyl) phosphine, according to some embodiments;

FIG. 3 shows a GC-MSD of 2-(2,4-Difluorophenylpyridine), according to some embodiments;

FIG. 4 shows a NMR of 2-(2,4-Difluorophenylpyridine), according to some embodiments;

FIG. 5 shows a HRMS of the prepared Ir Complex, according to some embodiments;

FIG. 6 shows powder x-ray diffraction plots for various MOFs, according to some embodiments;

FIG. 7 shows an absorption spectrum of X1 complex in PBS:methanol (8:2), according to some embodiments;

FIG. 8 shows an absorption spectrum of X2 complex in PBS:methanol (8:2), according to some embodiments;

FIG. 9 shows an absorption spectrum of X3 complex in PBS:methanol (8:2), according to some embodiments;

FIG. 10 shows an absorption spectrum of X4 complex in PBS:methanol (8:2), according to some embodiments;

FIG. 11 shows an absorption spectrum of X5 complex in PBS:methanol (8:2), according to some embodiments;

FIG. 12 shows an absorption spectrum of X6 complex in acetonitrile, according to some embodiments;

FIG. 13 shows an absorption spectra of X8 complex in PBS:methanol (8:2), according to some embodiments;

FIG. 14 shows a plot of phosphorescence quenching of X1 complex by 0% O2 (pure N2), according to some embodiments;

FIG. 15 shows a plot of phosphorescence quenching of X1 complex by 10% O2 in PBS+MeOH (8:2), according to some embodiments;

FIG. 16 show a plot of phosphorescence quenching of X1 complex by 20% O2 in PBS+MeOH (8:2), according to some embodiments;

FIG. 17 shows a plot of phosphorescence quenching of X1 complex by 100% O2 in PBS+MeOH (8:2), according to some embodiments;

FIG. 18 shows a plot of phosphorescence quenching of X2 complex by 10% O2 in PBS+MeOH (8:2), according to some embodiments;

FIG. 19 shows a plot of phosphorescence quenching of X2 complex by 20% O2 in PBS+MeOH (8:2), according to some embodiments;

FIG. 20 shows a plot of the phosphorescence quenching of X2 complex by 100% O2 in PBS+MeOH (8:2), according to some embodiments;

FIG. 21 shows a plot of the phosphorescence quenching of X3 complex by 10% O2 in PBS+MeOH (8:2), according to some embodiments;

FIG. 22 shows a plot of the phosphorescence quenching of X4 complex by 10% O2 in PBS+MeOH (8:2), according to some embodiments;

FIG. 23 shows a plot of the phosphorescent luminescent behavior of X5 complex, according to some embodiments;

FIG. 24 shows a plot of the phosphorescence quenching of X6 complex by 100% O2 in acetonitrile, according to some embodiments;

FIG. 25 shows a plot of the phosphorescence quenching of X8 complex by varying percentages of O2, according to some embodiments;

FIG. 26 shows a plot of the photoluminescence measurement of X12 MOF in THF solvent, according to some embodiments;

FIG. 27 shows a plot of the phosphorescence quenching of X12 MOF by 10% O2 in PPh3+THF solvent, according to some embodiments;

FIG. 28 shows a plot of the phosphorescence quenching of X12 MOF by 20% O2 in PPh3+THF solvent, according to some embodiments;

FIG. 29 shows a plot of the phosphorescence quenching of X12 MOF by 100% O2 in PPh3+THF solvent, according to some embodiments;

FIG. 30 shows a plot of the phosphorescence quenching of X12 MOF by 20% O2 in EtOH solvent, according to some embodiments;

FIG. 31 shows a plot of the phosphorescence quenching of X12 MOF by 100% O2 in EtOH solvent, according to some embodiments;

FIG. 32 shows a plot of the fit fluorescence intensity of X1 complex, according to some embodiments;

FIG. 33 shows a plot of, according to some embodiments;

FIG. 34 shows a plot of the quantum yield for X1 complex in PBS+MeOH, according to some embodiments;

FIG. 35 shows a plot of the quantum yield X12 MOF in PPh3+THF solvent, according to some embodiments.

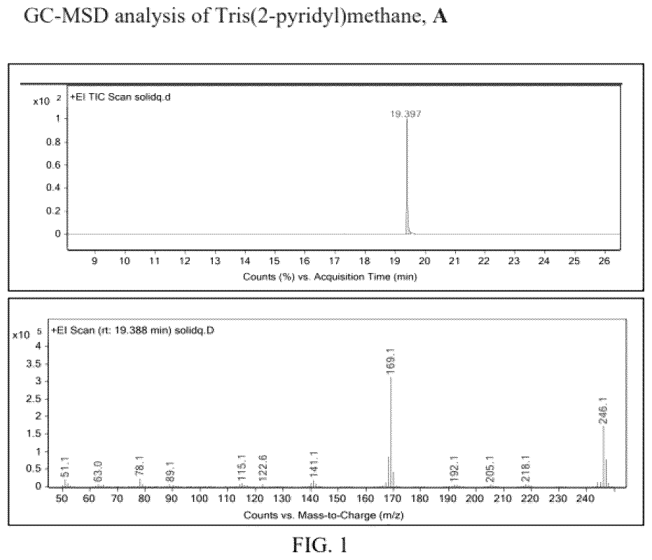

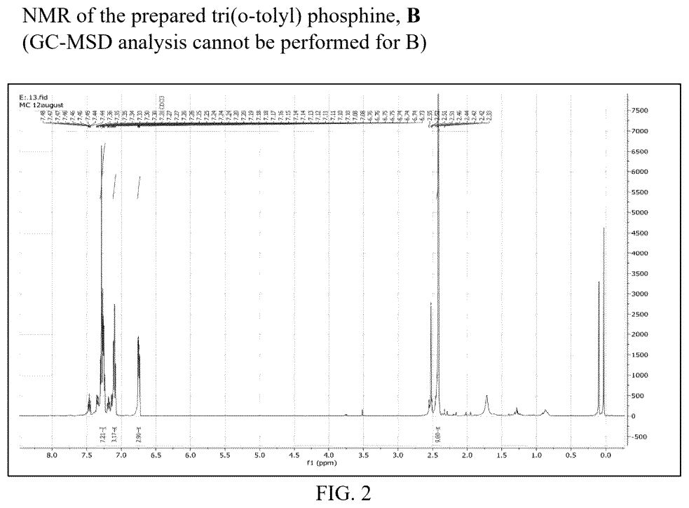

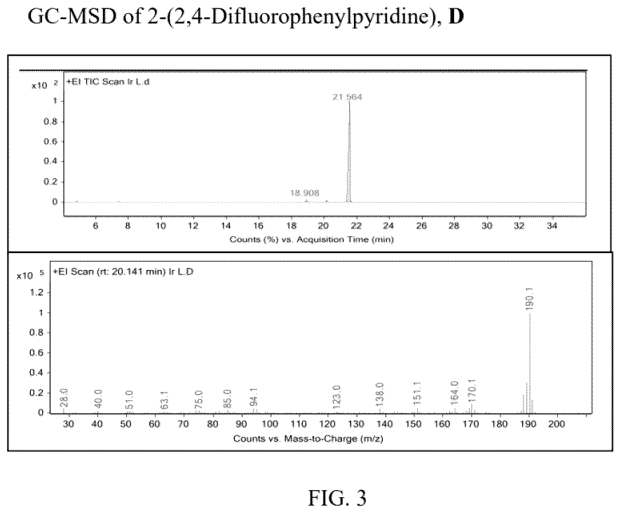

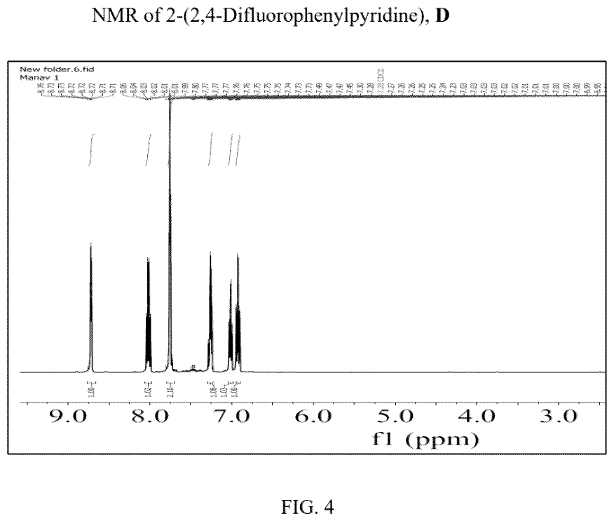

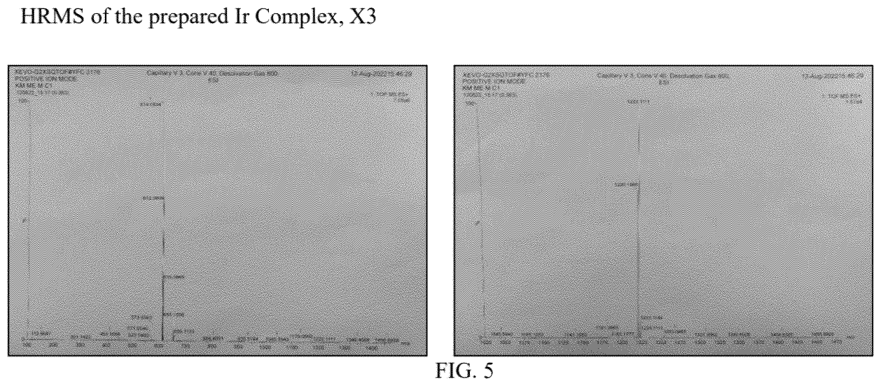

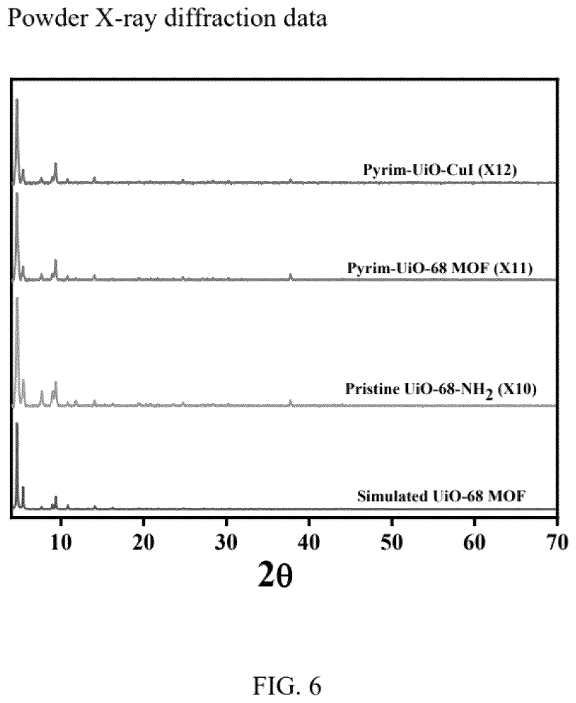

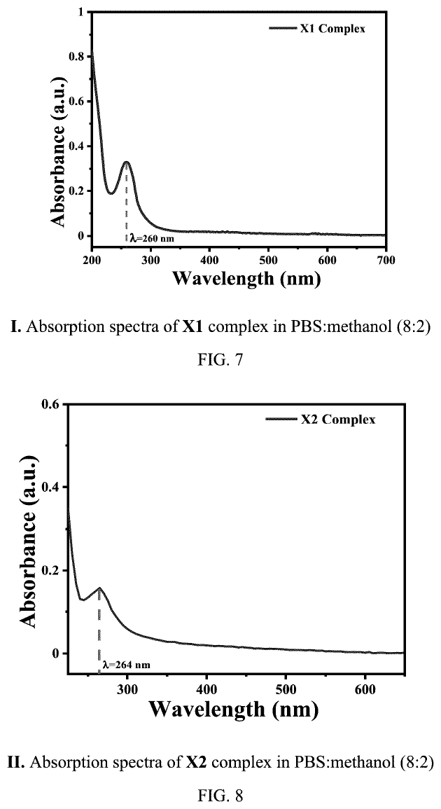

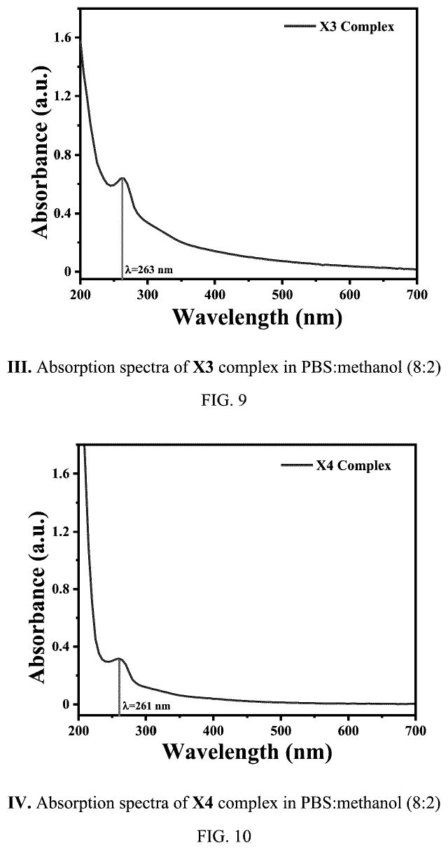

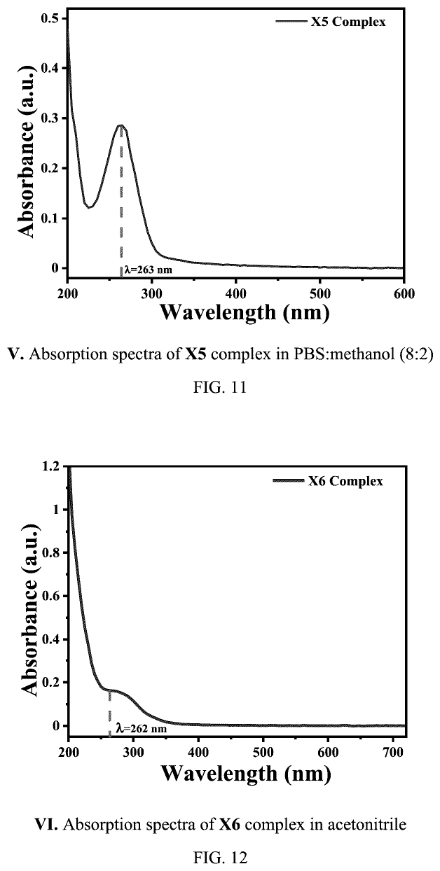

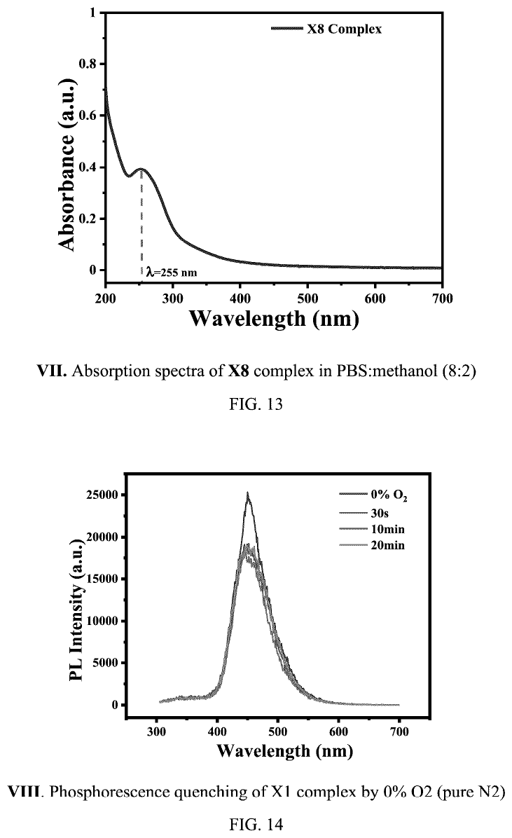

### DETAILED DESCRIPTION

Aspects of the disclosure generally relates to articles, compositions, and systems for oxygen detection in vitro or in vivo. In some embodiments, the disclosure relates to a metal complex, e.g., a compound comprising at least one metal-carbon bond. In some embodiments, the disclosure relates to metal organic frameworks (MOF). The MOFs comprise a plurality of metal clusters and a plurality of ligands that are coordinated with the plurality of metal clusters. In some embodiments, the disclosure relates to liposome-complexes (e.g., liposome-metal complexes and/or liposome-MOF complexes). In some embodiments, the metal complex, MOF, and/or liposome-complexes are configured to undergo photoluminescence following activation. Methods for in vitro and in vivo oxygen sensing and/or detecting intratumoral oxygen tensions using said articles, compositions, and system are also disclosed herein.

In some embodiments, the complex is a metal complex, for example, an organometallic compound. As used herein, the term organometallic compound refers to a coordination complex that has at least one metal-carbon bond. In some embodiments, the complex is a metal organic framework (MOFs). Without wishing to be bound by any particular theory, MOFs may be considered to be polymers of organometallic compounds.

In some embodiments, a complex comprises the structure shown in Formula (I):

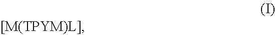

or a salt thereof, wherein M is Cu(I), Ir, Rh, Ag, Co, Fe, Ru, Ni, Zn, or Au; TPYM is Tris(2-pyridyl)methane, and L is a monophosphine ligand.

In some embodiments, the complex has the structure: [Cu(I)(TPYM)B], [TPYM=Tris(2-pyridyl)methane, B=monophosphine ligand]:

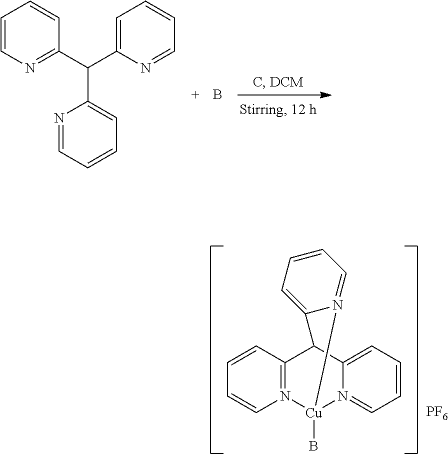

In some embodiments, the monophosphine ligand (B) is selected from the group consisting of:

| | | | Excitation | Emission | | |
|---|---|---|---|---|---|---|
| | Name of the | Structure of the | wavelength | wavelength | | |
| SNo. | ligand | ligand | (λₘₐₓ) | (λₘₐₓ) | Solvent | PL Behaviour |
| 1. | Tri(o-tolyl) phosphine | 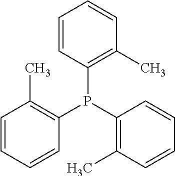 [SMILES: Cc1ccccc1P(c1ccccc1C)c1ccccc1C] | 260 nm | 443 nm | PBS + MeOH | PL Active, shows O₂ quenching |
| 2. | Methyldiphenyl phosphine | 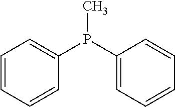 | 264 nm | 377 nm | PBS + MeOH | Does not show O₂ quenching |
| 3. | Tricyclohexyl- phosphine | 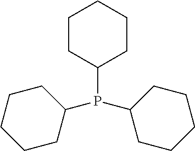 [SMILES: C1CCC(P(C2CCCCC2)C2CCCCC2)CC1] | 263 nm | 446 nm | PBS + MeOH | Does not show O₂ quenching |
| 4. | PCl₃ | 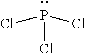 | 261 nm | 352 nm | PBS + MeOH | Does not show O₂ quenching |
| 5. | 1,2-bis (diphenylphosphino) benzene | 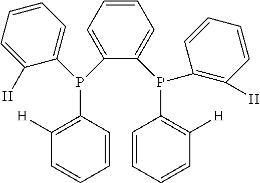 [SMILES: c1ccc(P(c2ccccc2)c2ccccc2P(c2ccccc2)c2ccccc2)cc1] | 262 nm | 343 nm | Acetonitrile | Does not show O₂ quenching |
| 6. | Triphenylphosphite | 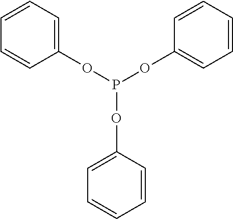 [SMILES: c1ccc(OP(Oc2ccccc2)Oc2ccccc2)cc1] | 263 nm | PL Inactive | PBS + MeOH | PL Inactive |

In some embodiments, a metal organic framework (MOF) comprises a plurality of metal clusters, wherein at least one metal cluster comprises a metal ion; and a plurality of ligands coordinating with the plurality of metal clusters, wherein the plurality of ligands comprises a triphenyldicarboxylic acid (TPDC)group or a triphenylphosphine group. In some embodiments, the plurality of ligands has any one of the following structures:

| Ligand | |
|---|---|
| label | Ligand Structure |
| X12 | 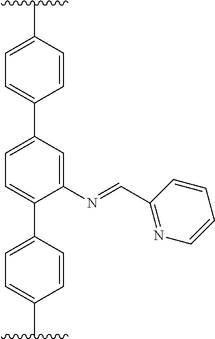 [SMILES: *c1ccc(-c2ccc(-c3ccc(*)cc3)c(/N=C/c3ccccn3)c2)cc1] |
| X11 | 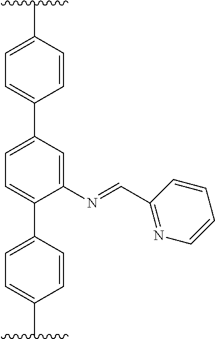 [SMILES: *c1ccc(-c2ccc(-c3ccc(*)cc3)c(/N=C/c3ccccn3)c2)cc1] |
| X9 | 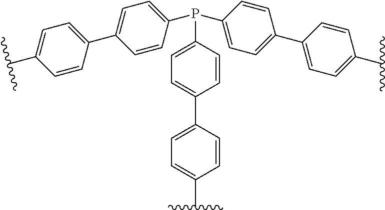 [SMILES: *c1ccc(-c2ccc(P(c3ccc(-c4ccc(*)cc4)cc3)c3ccc(-c4ccc(*)cc4)cc3)cc2)cc1] |

In some embodiments, the disclosure relates to articles, compositions, systems, and/or methods for oxygen detection. In some embodiments, the system comprises a complex. In some embodiments, the complex is a metal complex (e.g., an organometallic complex). In some embodiments, the complex is a metal organic framework. In some embodiments, the complex undergoes phosphorescence and/or fluorescence upon excitation, e.g., by exposure to light at a wavelength of between 300 nm to 800 nm. In preferred embodiments, the complex has an excitation wavelength of between 260 nm and 270 nm and emission wavelength between 340 nm and 450 nm. The complex exhibits a photoluminescent intensity of the emission wavelength that is at least partially quenched in a presence of molecular oxygen.

In some embodiments, exposure of the complex to oxygen, e.g., between 0% (0 mm Hg) and 100% (760 mm Hg) reduces (e.g., quenches) the intensity of the phosphorescence and/or fluorescence of the complex (e.g., as determined by fluorometer). In a preferred embodiment, molecular oxygen is present at a concentration of between 0.01 and 120 mmHg. Without wishing to be bound by any particular theory, it is generally believed that biosensing of molecular oxygen in vivo has significance across oxygen deficient (hypoxia) and excess oxygen (hyperoxia) environments, seen in tumors (cancerous and others) and cardiovascular disease respectively. In some embodiments, biosensing at close to normoxic conditions (20% or −120 mmHg O2) by the present system allows early detection of developing severely deficient disease microenvironments. Such systems became important during the Covid-19 pandemic and have relevance for detection of overdose (opioids, etc.). Some embodiments may thus be activated by short durations of exposure to the oxygen environment (30 seconds-2 minutes) for rapid detection via quenching of phosphorescence.

In some embodiments, an emission maxima of an complex is tunable, for example, by manipulating the structure of said complex. In some embodiments, the emission maxima is between the ultraviolet (UV) and infrared (IR) wavelengths of light (300-800 nm). In some embodiments, the emission maxima is greater than or equal to 300 nm, greater than or equal to 400 nm, greater than or equal to 500 nm, greater than or equal to 600 nm, greater than or equal to 700 nm, or greater than or equal to 800 nm. In some embodiments, the emission maxima is less than or equal to 800 nm, less than or equal to 700 nm, less than or equal to 600 nm, less than or equal to 500 nm, less than or equal to 400 nm, or less than or equal to 300 nm. Combinations of ranges are also possible (e.g., greater than or equal to 300 nm and less than or equal to 800 nm). Without wishing to be bound by any particular theory, the quantum yield, emission brightness & oxygen sensitivity are given at the target wavelength using spectrophotometry, lifetime fluorescence and other relevant methods. By mechanisms of emission like phosphorescence and Thermally Activated Delayed Fluorescence (TADF), internal quantum yield (IQE) up to 100% can be achieved compared to 25% with fluorescence. This can increase energy efficiency of the overall system. TADF also allows the use of heavy metal free emitters like iron (Fe) and copper (Cu), which brings down the cost of production.

In some embodiments, the metal complexes disclosed herein may be activated in vivo without light stimulus through the interaction of the metal complex structure with the biological environment depending on the composition, design and the desired application of the metal complex. There are several ways by which the metal complexes can be activated in vivo, and in some cases, more than one mechanism may occur:
a) Ligand Exchange: Metal complexes can undergo ligand exchange reactions in the presence of specific biomolecules or ligands present in the biological environment which results in the activation or alteration of the complex's properties; and/or,
b) Redox Chemistry: Metal complexes can undergo redox reactions within a biological system. They may interact with endogenous reducing agents or oxidising agents which results in the activation or transformation of the metal complex; and/or,
c) pH-Dependent Activation: The pH of biological environments can influence the reactivity of the metal complexes where changes in pH can alter the protonation state of ligands or affect the stability of the metal-ligand bonds. Subsequently, the metal complex may undergo activation or dissociation under specific pH conditions, resulting in changes in its behaviour or activity; and/or,
d) Metabolic Transformations: Metal complexes may undergo metabolic transformations within the body where the enzymatic processes can modify the structure of the complex or its ligands resulting in the activation or deactivation of the complex.

In some embodiments, the complex is a metal organic framework (MOF). In some embodiments, the MOF is loaded with a cargo, e.g., a drug. Without wishing to be bound by any particular theory, it is generally known in the art that MOFs have a high degree of chemical tunability (to interact with a number of biomolecules, pH environments etc.) and can modified be water soluble. MOFs are capable of being loaded with a higher quantity of cargo (e.g., drugs) or multiple cargos (e.g., more than one drug) with lower risk of burst release, relative to liposomes. The MOF described herein comprise a 3D structure with internal pores geometries (e.g., octahedral and tetrahedral). The solubility of the MOF may depend on the particle size. MOFs may show hydrophilic-hydrophobic interactions as the metal sites in the MOF are hydrophilic and the linker species are hydrophobic. Also, the MOFs may have strong metal-ligand bonds which are difficult to hydrolyse. On the basis of these interactions, the MOF structure can be further modified to encapsulate the cargo within one or more pores. However, if the drug size is large, then the hydrophilicity-hydrophobicity nature of MOFs may not play a role in encapsulating the drugs. Additionally, MOFs are biodegradable in some embodiments. Thus, certain MOFs have applicability as therapeutics and/or diagnostics (e.g., theranostics), as well as in manufacturing of technologies including but not limited to separation/purification, catalysis and storage of materials & energy due to its highly porous structure.

In some embodiments, MOF disclosed herein are embedded within a lipid bilayer of a liposome, as described in “Metal-organic frameworks embedded in a liposome facilitate overall photocatalytic water splitting” Nat. Chem. 2021; 13(4): 358-366. Without wishing to be bound by any particular theory, it is believed that embedding the MOF within the lipid bilayer allows the MOF to be in contact with the surrounding environment (e.g., tissue microenvironment) for oxygen sensing under certain conditions, e.g., by phosphorescence quenching of any one of the complexes disclosed herein.

In some embodiments, the liposome-MOF complex comprises an oxygen sensing MOF (e.g., a Cu-MOF) conjugated to the lipid bilayer of a liposome loaded with a cargo, wherein said liposome is made of light sensitive lipids as described elsewhere herein. In some embodiments, the light sensitive lipid is selected so that it undergoes photo-triggering at the emission wavelength of the Cu-MOF, resulting in rupture of the liposome followed by cargo release, upon hypoxic environment sensing by the Cu-MOF.

In some embodiments, MOFs comprise the formula: Zr6O4(OH)4(LMx)6 where L is a nitrogen-based ligand with triphenyldicarboxylic acid (TPDC) as the bridging linker and M can be any metal (Cu, Co, Ag, Ir etc.) with Mx as the catalyst loading which can be calculated by ICP-OES.

In some embodiments, a copper-pyridylimine-functionalized MOF (pyrim-MOF-Cu) has the structure:

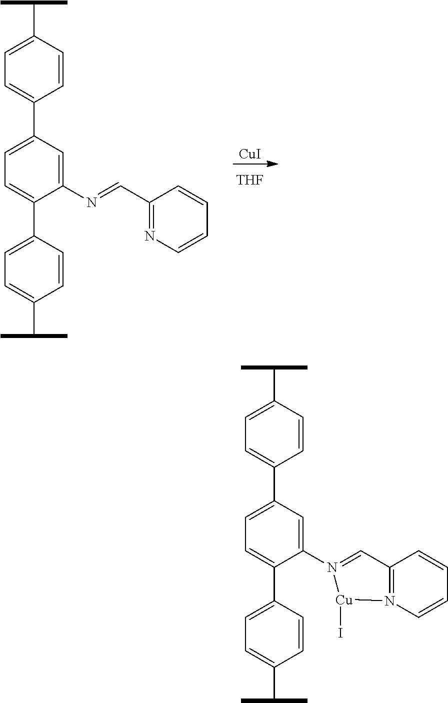

In some embodiments, the solubility of the MOF depends on the particle size. The MOF shows hydrophilic-hydrophobic interactions as the metal sites in the MOF are hydrophilic and the linker species are hydrophobic. Also, the MOFs have highly strong metal-ligand bonds which are difficult to hydrolyse.

In some embodiments, the MOF disclosed herein may be activated in vivo without light stimulus through the interaction of the MOF structure with the biological environment depending on the composition, design and the desired application of the MOF. There are several ways by which the MOFs can be activated in vivo:
a) Ion Exchange: MOFs when exposed to specific molecules or ions in the biological system, can undergo ion exchange processes. When the MOF comes into contact with metal ions in the solution or when it interacts with ions present in the biological fluids, there occurs an exchange of metal ions in the MOF. This ion exchange process results in the modification of MOF's composition, structure and properties resulting in the altered reactivity or targeted release of encapsulated substances; and/or,
b) Biodegradation: Some MOFs can be designed biodegradable as they can be broken down in a biological environment. This degradation process may be triggered by either the presence of certain enzymes or other biologically relevant factors. As the MOF degrades, it can release the encapsulated substances or undergo structural changes enabling the desired biological activity; and/or,
c) pH-Dependent Responses: MOFs can exhibit pH-dependent behaviour where changes in the acidity or basicity of the surrounding environment can influence their properties. Also, variations in pH can affect the stability of the MOF structure or trigger the release of loaded molecules; and/or,
d) Enzymatic Interactions: MOFs may interact with specific enzymes present in the biological system, where the enzymes may bind to the MOF surface or catalyse reactions within the MOF structure. These interactions can trigger changes in the MOF's properties like targeted drug delivery, controlled release and activation of catalytic processes.

In some embodiments, the complexes (e.g., metal complexes and/or MOFs) herein undergo Thermally Activated Delayed Fluorescence (TADF). The term TADF may refer to the ability of a molecular species in a non-emitting excited state to incorporate surrounding thermal energy to change states and only then undergo light emission. It is a structural property requiring no external stimuli.

In some embodiments, the complex (e.g., organometallic complex and/or MOF) comprises any one of the following metal center (M) and ligand systems (L).

| | |
|---|---|
| Metal | Sc, Y, La, Ti, Zr, Hf, V, Nb, Ta, Cr, Mo, W, Mn, Tc, Re, Fe, Ru, Os, Co, Rh, |
| center (M) | Ir, Ni, Pd, Pt, Cu, Ag, Au, Zn, Cd, Hg, Al, Ga, In, Tl, Si, Ge, Sn, Pb, P, As, Sb, |
| | Bi, S, Se, Te, Po, Cl, Br, I, At, lanthanides, actinides, and 2H |
| Ligand | 1. Tri(o-tolyl)phosphine |
| system (L) | 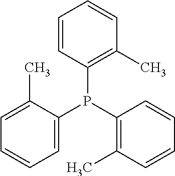 [SMILES: Cc1ccccc1P(c1ccccc1C)c1ccccc1C] |
| | 2. Tris(2-pyridyl)methane |
| | 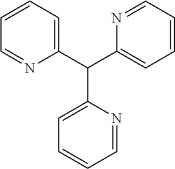 [SMILES: c1ccc(C(c2ccccn2)c2ccccn2)nc1] |
| | 3. 2-(2,4-Difluorophenylpyridine) |
| | 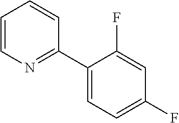 [SMILES: Fc1ccc(-c2ccccn2)c(F)c1] |
| | 4. (2S,2′S,2″S)-1,1′,1″-nitrilotris(propan-2-ol) |
| | 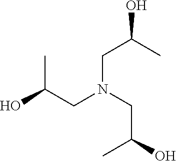 |
| | 5. 1,4,7-trimethyl-1,4,7-triazonane |
| | 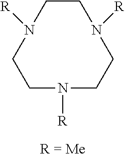 |
| | 6. Tri(1H-pyrazol-1-yl)methanesulfonate |
| | 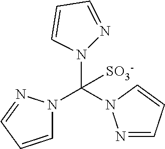 |
| | 7. (4S,4′S)-2,2′-(2-((R)-4-isopropyl-4,5-dihydrooxazol-2- |
| | yl)propane-1,2-diyl)bis(4-isopropyl-4,5- |
| | dihydrooxazole) |
| | 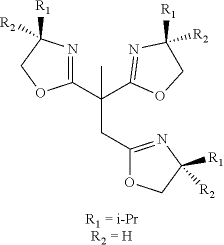 |
| | 8. |
| | 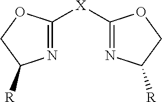 |
| | a) When X = C |
| | (i) When R = i-Pr then bis((S)-4-isopropyl-4,5- |
| | dihydrooxazol-2-yl)methane |
| | (ii) When R = Ph then bis((S)-4-phenyl-4,5- |
| | dihydrooxazol-2-yl)methane |
| | (iii) When R = Me then bis((S)-4-methyl-4,5- |
| | dihydrooxazol-2-yl)methane |
| | (iv) When R = t-Bu then bis((S)-4-(tert-butyl)-4,5- |
| | dihydrooxazol-2-yl)methane |
| | b) When X = N |
| | (i) When R = i-Pr then (S)-bis((S)-4-isopropyl-4,5- |
| | dihydrooxazol-2-yl)amine |
| | (ii) When R = Ph then (S)-bis((S)-4-phenyl-4,5- |
| | dihydrooxazol-2-yl)amine |
| | (iii) When R = Me then (S)-bis((S)-4-methyl-4,5- |
| | dihydrooxazol-2-yl)amine |
| | (iv) When R = t-Bu then (S)-bis((S)-4-(tert-butyl)-4,5- |
| | dihydrooxazol-2-yl)amine |
| | 9. Tris(((S)-4-isopropyl-4,5-dihydrooxazol-2-yl)methyl)amine |
| | 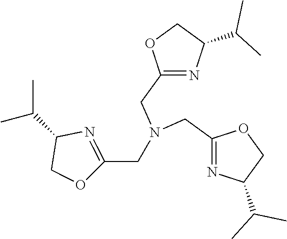 [SMILES: CC(C)[C@H]1COC(CN(CC2=N[C@@H](C(C)C)CO2)CC2=N[C@@H](C(C)C)CO2)=N1] |
| | 10. Tris(((S)-4-benzyl-4,5-dihydrooxazol-2-yl)methyl)amine |
| | 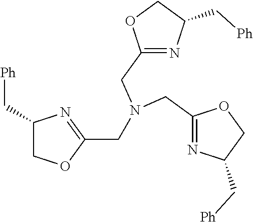 [SMILES: c1ccc(C[C@H]2COC(CN(CC3=N[C@@H](Cc4ccccc4)CO3)CC3=N[C@@H](Cc4ccccc4)CO3)=N2)cc1] |
| | 11. Tris(((S)-4-methyl-4,5-dihydrooxazol-2-yl)methyl)amine |
| | 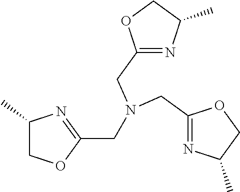 [SMILES: C[C@H]1COC(CN(CC2=N[C@@H](C)CO2)CC2=N[C@@H](C)CO2)=N1] |
| | 12. Tri(1H-1,2,4-triazol-1-yl)phosphine oxide |
| | 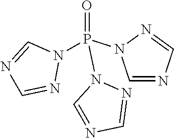 [SMILES: O=P(n1cncn1)(n1cncn1)n1cncn1] |
| | 13. (1S,2S)-N¹,N²-dimethyl-1,2-diphenylethane-1,2-diamine |
| | 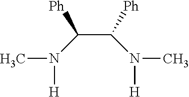 |
| | 14. (S)-4-isopropyl-2-(1,2,3,4-tetrahydroquinolin-8-yl)-4,5-dihydrooxazole |
| | 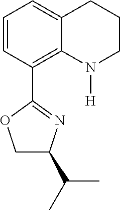 [SMILES: CC(C)[C@H]1COC(c2cccc3c2NCCC3)=N1] |
| | 15. (S)-2-(2-(diphenylphosphino)phenyl)-4-isopropyl-4,5-dihydrooxazole |
| | 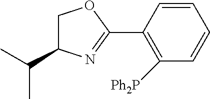 |
| | 16. Bis(((R)-4-phenyl-4,5-dihydrooxazol-2-yl)methyl)amine |
| | 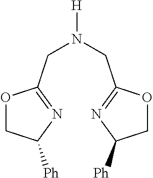 [SMILES: c1ccc([C@@H]2COC(CNCC3=N[C@H](c4ccccc4)CO3)=N2)cc1] |
| | 17. 1,2-bis((S)-4-isopropyl-4,5-dihydrooxazol-2-yl)benzene |
| | 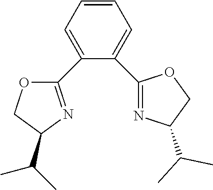 [SMILES: CC(C)[C@H]1COC(c2ccccc2C2=N[C@@H](C(C)C)CO2)=N1] |
| | 18. 2-((R)-4-isopropyl-4,5-dihydrooxazol-2-yl)-2′-((S)-4- |
| | isopropyl-4,5-dihydrooxazol-2-yl)-1,1′-biphenyl |
| | 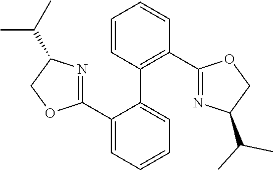 [SMILES: CC(C)[C@@H]1COC(c2ccccc2-c2ccccc2C2=N[C@@H](C(C)C)CO2)=N1] |
| | 19. (4R,4′R)-2,2′-((phenylphosphinediyl)bis(methylene)) |
| | bis(4-phenyl-4,5-dihydrooxazole) |
| | 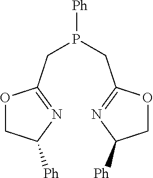 [SMILES: c1ccc([C@@H]2COC(CP(CC3=N[C@H](c4ccccc4)CO3)c3ccccc3)=N2)cc1] |
| | 20. Bis((3aR, 8aS)-8,8a-dihydro-3aH-indeno[1,2-d]oxazol-2-yl)methane |
| | 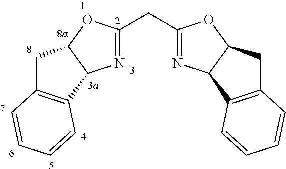 [SMILES: c1ccc2c(c1)C[C@@H]1OC(CC3=N[C@@H]4c5ccccc5C[C@@H]4O3)=N[C@H]21] |
| | 21. Bis((4R,5S)-4,5-diphenyl-4,5-dihydrooxazol-2-yl)methane |
| | 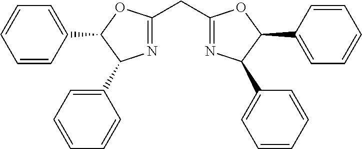 [SMILES: c1ccc([C@H]2N=C(CC3=N[C@H](c4ccccc4)[C@H](c4ccccc4)O3)O[C@H]2c2ccccc2)cc1] |
| | 22. Bis((S)-5,5-dimethyl-4-phenyl-4,5-dihydrooxazol-2-yl)methane |
| | 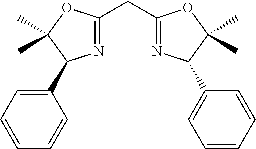 |
| | 23. Bis((4S,5R)-4,5-bis(4-(tert-butyl)phenyl)-4,5-dihydrooxazol-2-yl)methane |
| | 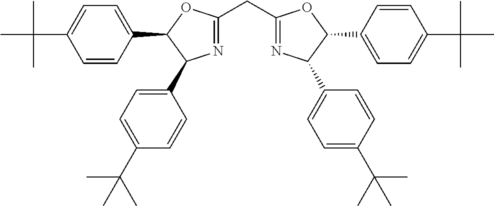 [SMILES: CC(C)(C)c1ccc([C@H]2OC(CC3=N[C@@H](c4ccc(C(C)(C)C)cc4)[C@@H](c4ccc(C(C)(C)C)cc4)O3)=N[C@H]2c2ccc(C(C)(C)C)cc2)cc1] |
| | 24. Bis((4S,5R)-4,5-di(naphthalen-2-yl)-4,5-dihydrooxazol-2-yl)methane |
| | 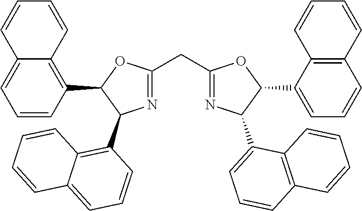 [SMILES: c1ccc2c([C@H]3OC(CC4=N[C@@H](c5cccc6ccccc56)[C@@H](c5cccc6ccccc56)O4)=N[C@H]3c3cccc4ccccc34)cccc2c1] |
| | 25. DuanPhos |
| | 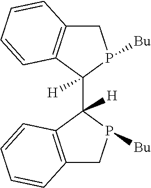 |
| | 26. TangPhos |
| | 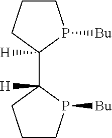 |
| | 27. Phanephos |
| | 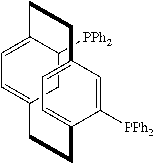 |
| | 28. Binapine |
| | 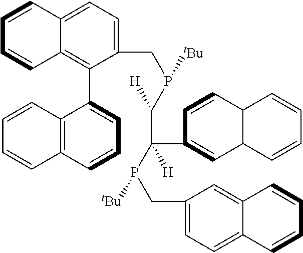 |
| | 29. SpiroPhos |
| | 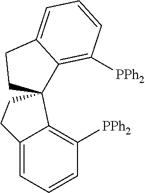 |
| | 30. QuinoxP* |
| | 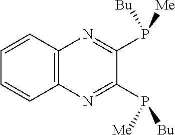 |
| | 31. Duphos |
| | 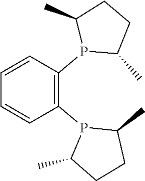 [SMILES: C[C@H]1CC[C@H](C)P1c1ccccc1P1[C@@H](C)CC[C@@H]1C] |
| | 32. Binaphane |
| | 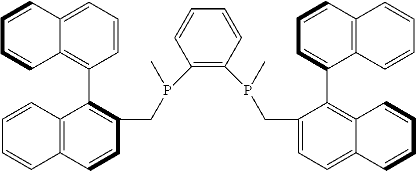 |
| | 33. Chiraphos |
| | 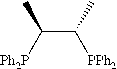 |
| | 34. (2S,4S)-2,4-Bis(diphenylphosphino)pentane |
| | 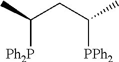 |
| | 35. DIPAMP |
| | 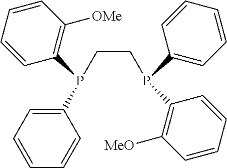 [SMILES: COc1ccccc1[P@](CC[P@](c1ccccc1)c1ccccc1OC)c1ccccc1] |
| | 36. (R,R)- DIOP |
| | 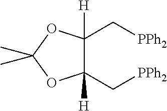 |
| | 37. (S)-(R)-bppfa |
| | 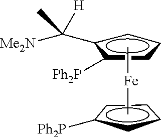 |
| | 38. (R,R)-Et-FerroTANE |
| | 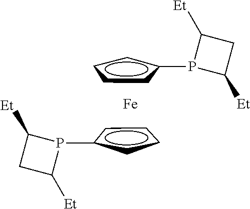 |
| | 39. WALPHOS |
| | 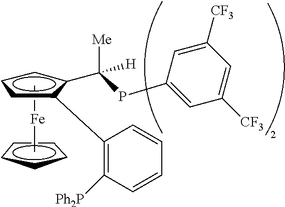 |
| | 40. Josiphos |
| | 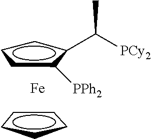 |
| | 41. (R,R)-Trost ligand |
| | 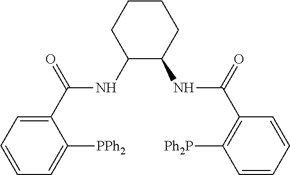 |
| | 42. (S)-MAP |
| | 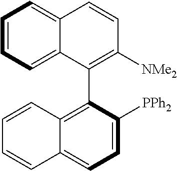 |
| | 43. SIPHOS |
| | 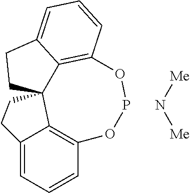 |
| | 44. Phosphoramidite |
| | 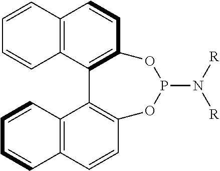 |
| | 45. MeO-MOP |
| | 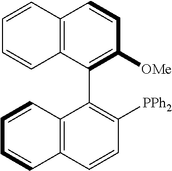 |
| | 46. Quinap |
| | 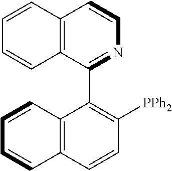 |

In some embodiments, the ligands are nitrogen-based ligands. In some embodiments, the nitrogen-based ligands comprise any one of the following structures:

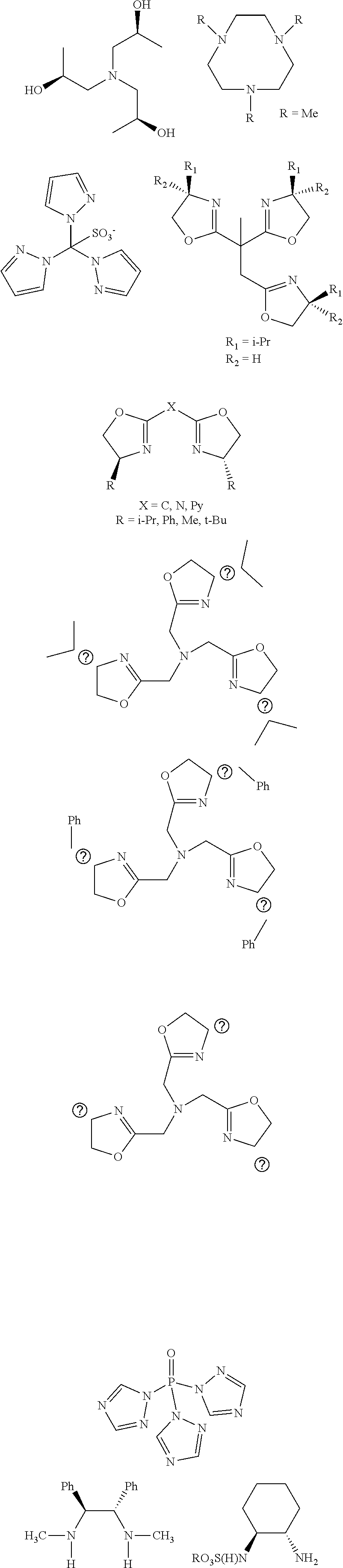

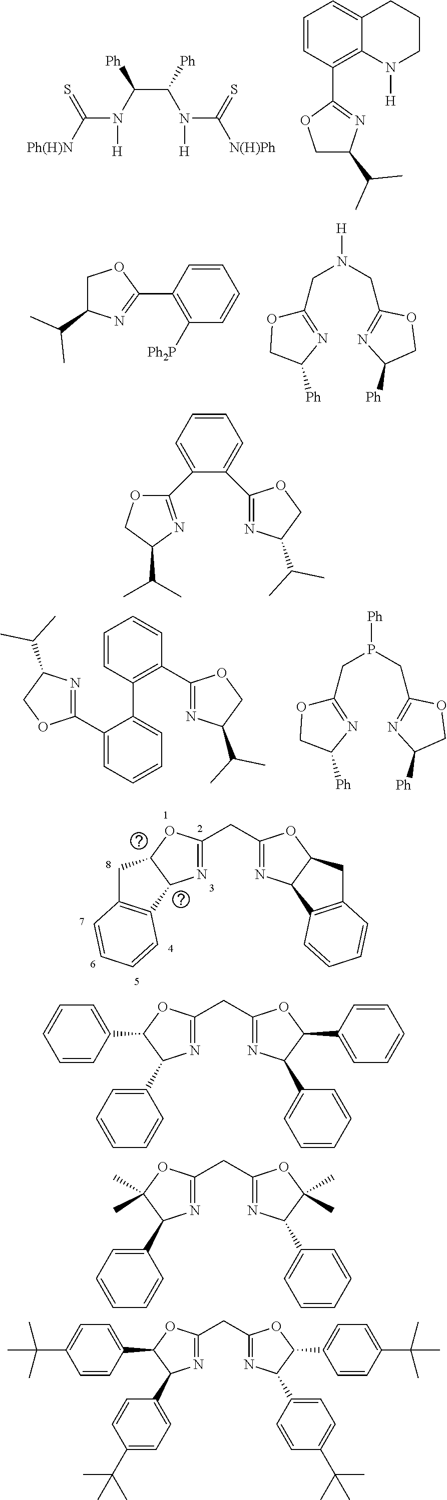

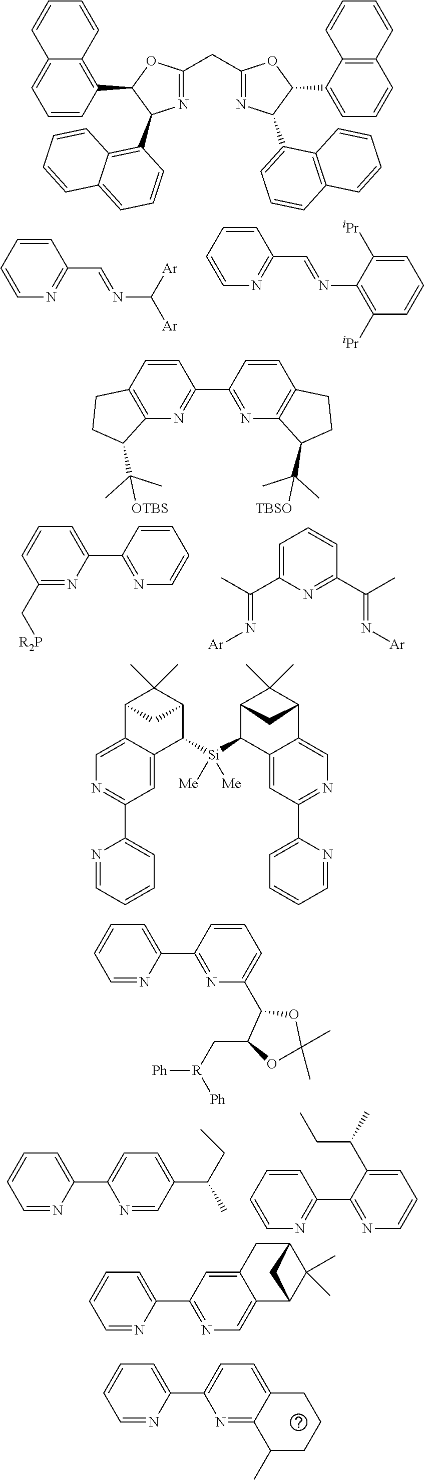

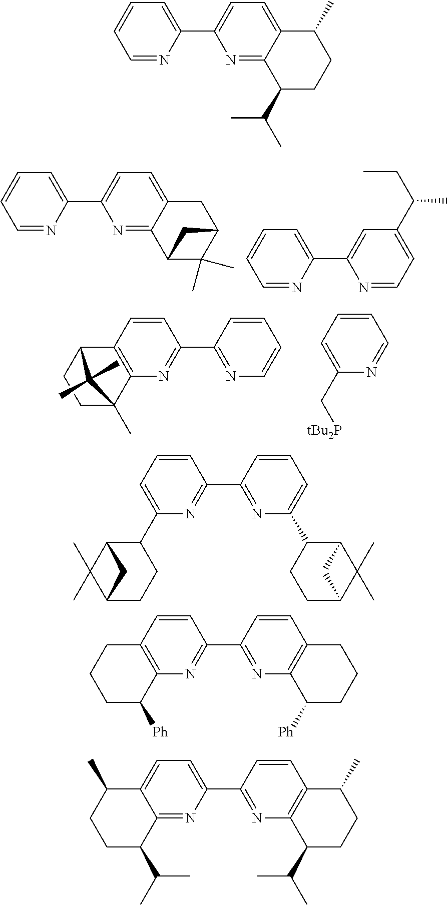

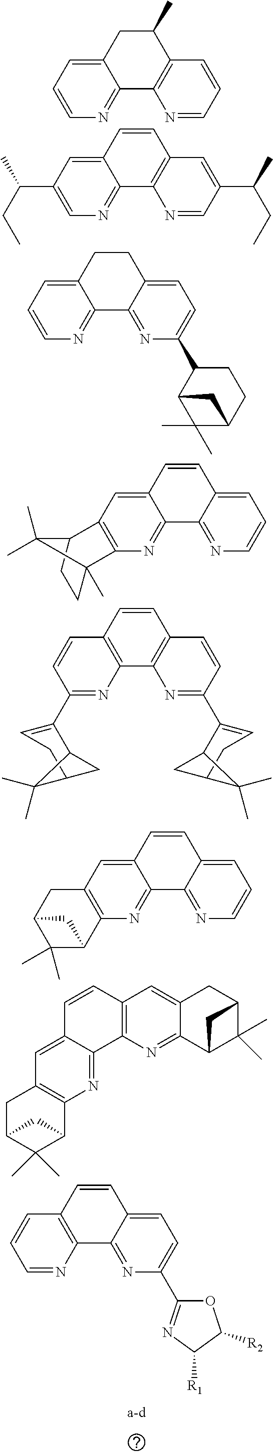

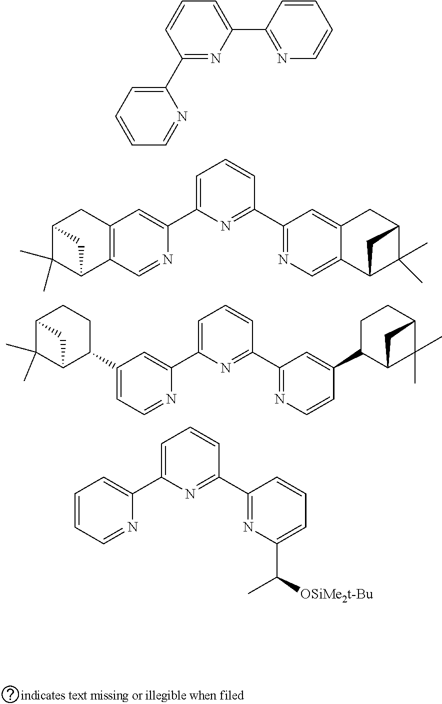

In some embodiments, the ligands are phosphorous-based ligands. In some embodiments, the phosphorus-based ligands comprise any one of the following structures:

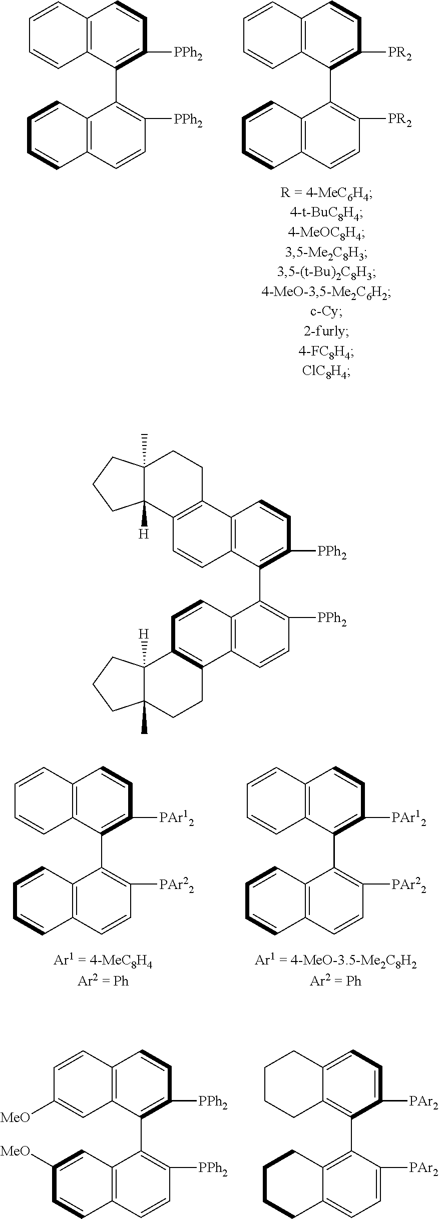

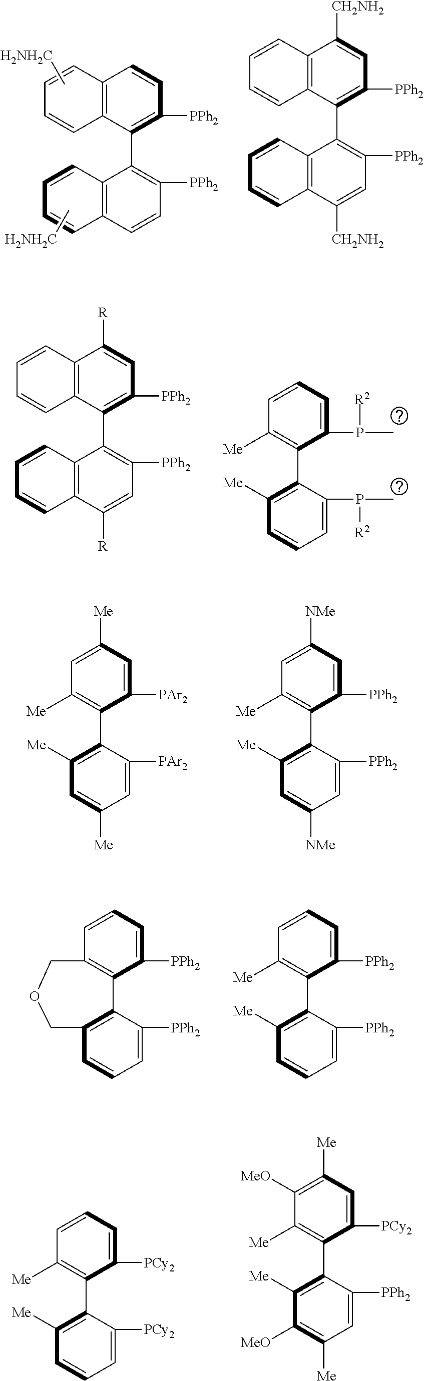

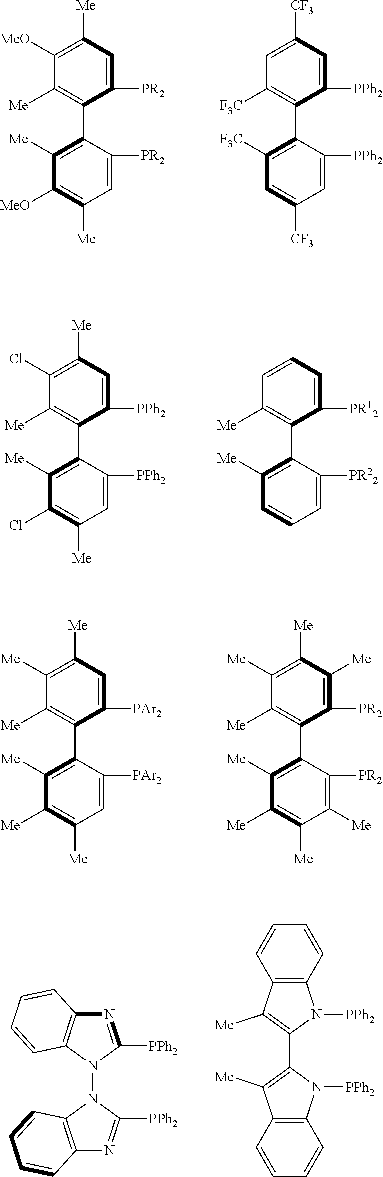

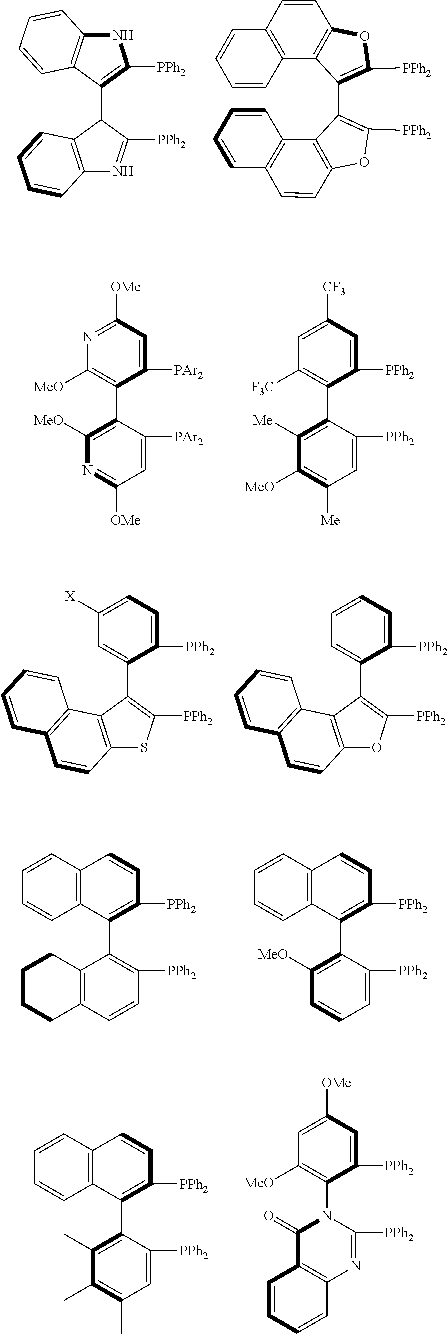

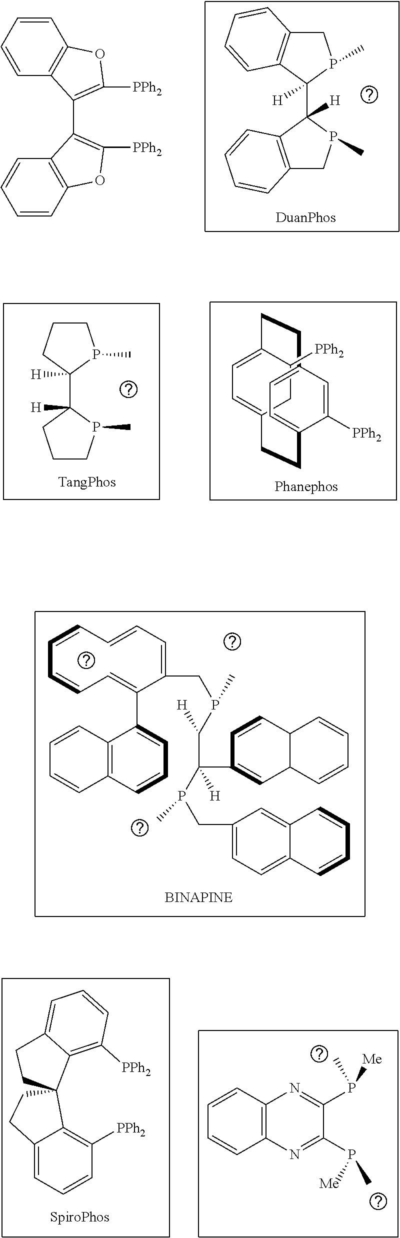

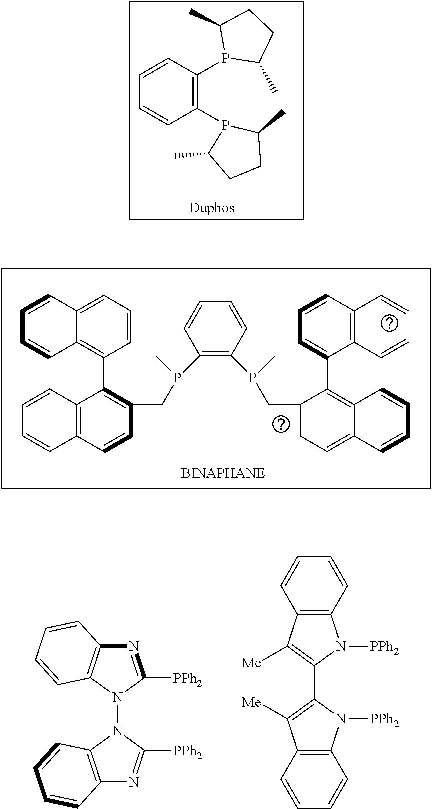

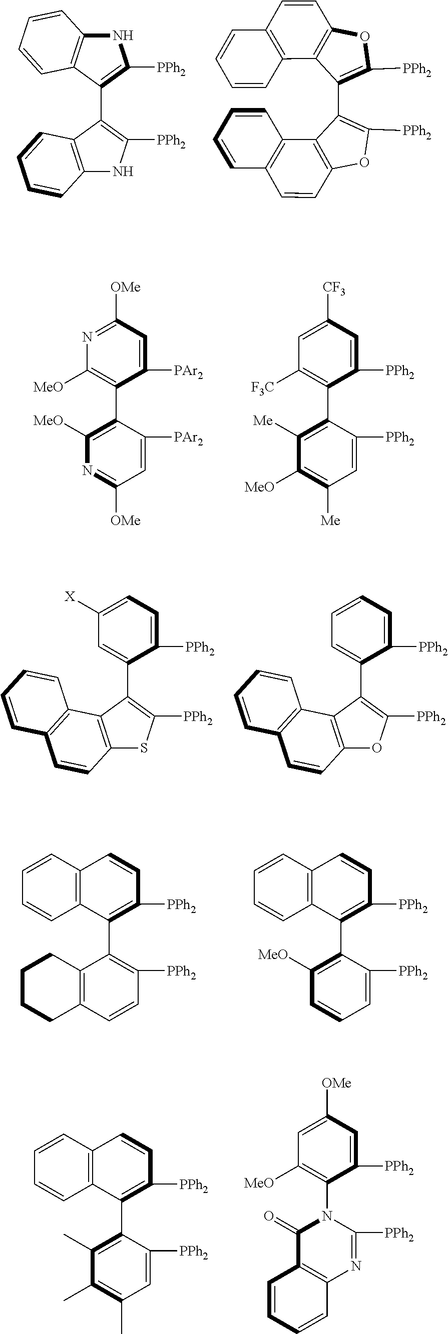

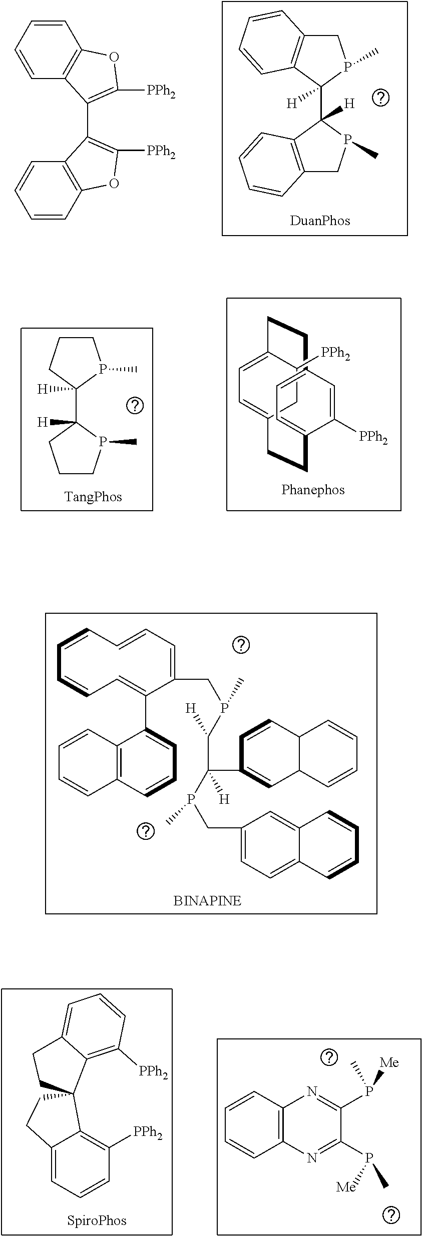

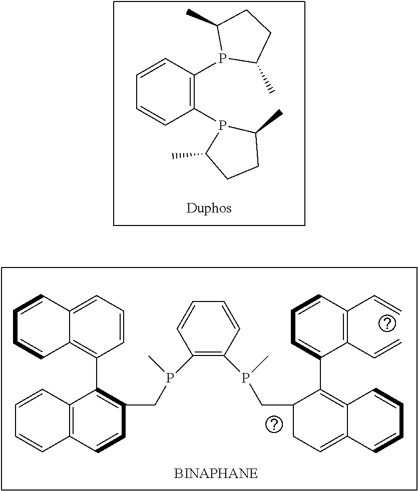

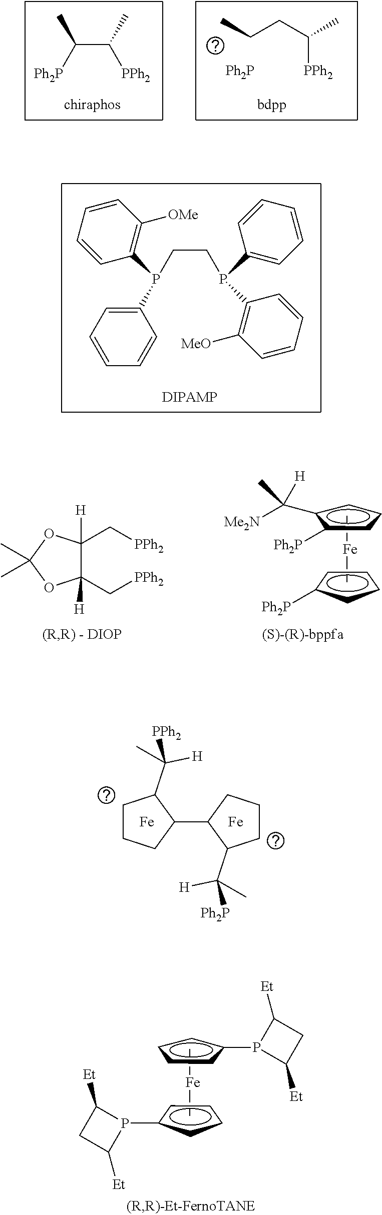

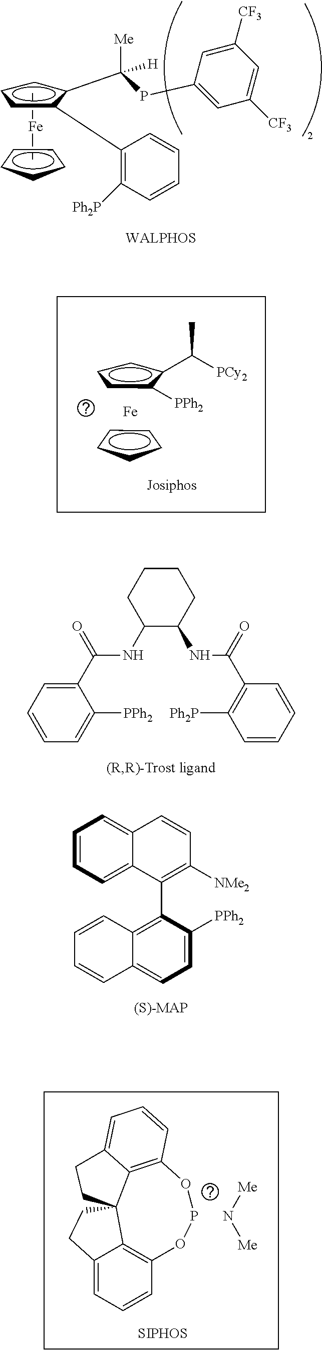

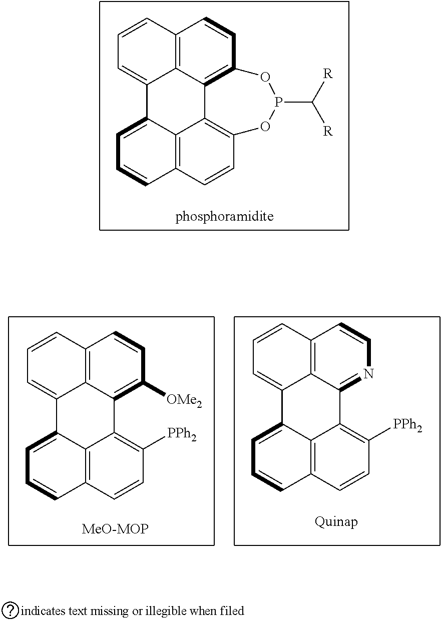

Other aspects of the disclosure relate to articles, compositions, systems, and/or methods comprising liposome-complexes (e.g., liposome-MOFs or liposome-metal complexes). In some embodiments, the liposome is a stimuli responsive liposome, e.g., to enable release of a cargo (e.g., a drug). In some embodiments, the release mechanism is based on the emission maxima of any one of the complexes disclosed herein. In some embodiments, the release mechanism is triggered when exposed to a low oxygen environment, e.g., an oxygen concentration of less than 120 mm Hg. Without wishing to be bound by any particular theory, it is generally recognized in the art that liposomes can be designed so they undergo at least any one or more of the following in response to a photothermal/photochemical stimulus: fragmentation, polymerization, and/or morphological changes. This allows, for example, various cargos to be encapsulated and delivered on demand. For instance, in some embodiments, drugs (e.g., chemotherapies, etc.) may be released when exposed to hypoxic cancerous tumors, additionally, cholesterol reducing statins may be delivered directly to high atherosclerotic plaque regions immediately after detection of hyperoxic conditions by the complex-liposome system. Stearic stabilization allows the liposome to circulate in the body for longer durations of time up to 24 hours to “wait” for the optimal disease microenvironment where it can deliver the drug or detect the onset of overdose conditions, as the case may be.

In some embodiments, the liposome is a phototriggerable liposome such as those described in “Phototriggerable Liposomes: Current Research and Future Perspectives” Pharmaceutics. 2014; 6(1):1-25. In some embodiments, the photosensitive component is verteporfin, a photosensitive lipid, clorin e6, a metal ion, photoprins, phthalocyanines, or porphyrin phthalocyanine. In some embodiments, the photosensitive lipid is plasmalogen, 1,2-bis(tricosa-10,12-diynoyl)-sn-glycero-3-phosphocholine (DC_{8,9}PC), DPPE-DVBA, Bis-Azo PC, or bis-sorbyl phosphatidylcholine (Bis-SorBbPC). In some embodiments, the liposome comprises phospholipids comprising Azobenzene groups such as Bis Azo PC. Without wishing to be bound by any particular theory, it is generally believed that such composition are capable of releasing a cargo upon exposure to visible light in the region of 470 nm, which is close to the emission wavelength (443 nm) of the complexes disclosed herein.

In some embodiments, a complex is embedded and/or encapsulated within the liposome. For example, in some embodiments, the complexes are embedded within the lipid bilayer of the liposome (e.g., hydrophobic complexes). In some embodiments, the complexes are encapsulated within the interior of the liposome (e.g., hydrophilic complexes). In some embodiments, the complexes are conjugated to the liposome. The complexes may be conjugated to an interior and/or exterior surface of the liposome; alternatively, or additionally, the complexes may be conjugated within the bulk of the lipid bilayer. The mode of conjugation may be chemical or physical. Thus, in some embodiments, the complexes are chemically conjugated to the liposome; and in some embodiments, the complexes are physically complexed (e.g., non-chemically conjugated) to the liposomes (e.g., via electrostatic bonding, hydrogen bonding, van der Waals interactions, etc.).

One or more mechanism may be used to rupture the liposome, for example to release the encapsulated cargo (e.g., drug). In some embodiments, the liposome may be ruptured by MOFs via any one of the following mechanisms:
(a) Mechanical Disruption: Some MOFs possess sharp edges or rough surfaces that can physically interact with the liposome bilayer. These interactions may result in mechanical disruptions such as physical abrasion or puncturing of the liposome membrane by the MOF particles. The physical disruption of the liposome structure can cause leakage or release of the encapsulated contents; and/or,
(b) Competitive Adsorption: MOFs can compete with lipids in the liposome membrane for space and interactions. Depending on the specific properties of the MOF and the lipids, MOFs may adsorb onto the liposome surface disrupting the lipid bilayer organisation. This disruption can compromise the integrity of the liposome and lead to the release of the encapsulated content; and/or,
(c) Lipid Extraction: Some MOFs possess metal sites or functional groups that can interact with lipid molecules. These interactions may lead to the extraction of lipids from the liposome membrane disturbing the lipid bilayer's structural integrity. As a result, the liposome can become destabilised and undergo rupture or leakage; and/or
(d) Reactive Species Generation: Some MOFs can generate reactive species such as reactive oxygen species (ROS) within their structure. These reactive species can diffuse from the MOF surface and interact with the liposome membrane. The oxidative stress induced by the ROS can disrupt the lipid bilayer resulting in liposome rupture and release of the encapsulated contents.

In some embodiments, the metal complexes disclosed herein are capable of disrupting the liposome structure via one or more of the following mechanisms:
(a) Metal complexes having high affinity for lipid membrane can interact with the lipids containing the liposome membrane resulting in changes in the membrane's integrity which leads to rupture or leakage. Metal complexes can disrupt liposomes through the insertion of their hydrophobic regions into the lipid bilayer. The hydrophobic parts of the metal complex like the organic ligands or hydrophobic metal centers interact with the hydrophobic tails of the lipids resulting in the perturbation of the lipid packing and destabilization of the membrane. This disruption may result in the formation of transient pores or defects in the liposome membrane leading to leakage of encapsulated contents; and/or
(b) Metal complexes can also interact with the head groups of the lipids wherein the metal complexes having polar or charged moieties interact with the polar headgroups of the lipids, thereby altering their organization and destabilising the liposome membrane. These interactions in turn can disrupt the lipid bilayer structure resulting in the membrane rupture.

In some embodiments, photons are not directly generated during the rupturing process of liposomes by metal complexes. The metal complex having phosphorescent or fluorescent properties can emit photons upon excitation. The emission in turn can occur before and/or after the rupture of the liposomes. The metal complexes exhibiting phosphorescent properties can be incorporated into liposomes as oxygen sensing probe. During the interaction of the metal complex with the molecular oxygen, the phosphorescent emission may be quenched, indicating the presence of oxygen. Hence, the emission of photons may occur as a result of the excited-state metal complex interacting with oxygen and not directly due to rupture of liposomes.

In some embodiments, the cargo is any suitable pharmaceutical agent known to the skilled artisan, including but not limited to, small molecules, peptides, proteins, nucleic acids, polynucleotides (DNA, RNA), RNA therapeutics (siRNA, mRNA, etc.), chemotherapeutic agents (e.g., thoxysuccinato-cisplatin (ESCP), TPT, busulfan, doxorubicin, camptothecin, diagnostic imaging agents (e.g., calcein), or other pharmaceutical agents. In some embodiments, the cargo is Narcan.

In some embodiments, the liposome-MOFs disclosed herein are configured to detect oxygen via one or more of the following methods:
a) Luminescent MOFs with oxygen-sensitive probes: Some MOFs can incorporate oxygen-sensitive probes within their structure. These probes comprise luminescent molecules that exhibit changes in their emission properties depending on the oxygen concentration in their environment. The luminescent molecules can be immobilized within the MOF pores or get attached to the MOF surface.
b) Quenching effects: The luminescent molecules in the MOF structure can be designed to undergo quenching or dequenching in the presence of oxygen. In the absence of oxygen, the luminescent molecules may not get quenched and exhibit strong emission. Whereas in the presence of oxygen, it can interact with the luminescent molecules and can quench their emission resulting in a decrease in the luminescence intensity.
c) Implantation or administration of MOFs: MOFs can be utilized for in vivo oxygen sensing by administrating them at the desired location within the body. MOFs can be encapsulated in the matrices or biocompatible coatings and can be implanted in tissues and organs. They can also be incorporated into nanoparticles for targeted delivery to specific sites.

In some embodiments, the liposome-metal complexes disclosed herein are configured to detect oxygen via one or more of the following methods:
(a) Metal complexes can be used in vivo for oxygen sensing in the absence of light. An example of metal complexes used for the same is based on phosphorescent probes. These probes may comprise a transition metal center coordinated to ligands that modulate the photophysical properties of the metal complex. The ligands may be chosen in such a way that the oxygen molecules can interact with the metal center, which may result in changes in the phosphorescent properties of the complex; and/or,
(b) In the absence of oxygen, the metal complexes may exhibit long phosphorescence lifetimes. But, in the presence of molecular oxygen, phosphorescence quenching may occur due to dynamic collision between the excited state metal complex and the oxygen molecules where the rate of quenching may be directly related to the concentration of oxygen in the surrounding environment; and/or,
(c) In order to detect the oxygen levels in vivo, the metal complexes can be incorporated into the biocompatible materials or can be conjugated to biomolecules like proteins.

Oxygen-sensing probes may be developed which can be linked to specific proteins or can be targeted to specific cellular compartments which allows for spatial monitoring of oxygen levels in various biological systems. This may be performed by time-resolved spectroscopy. The oxygen concentration can be determined by measuring the decay time of the phosphorescence emitted by the metal complex which enables the quantification of oxygen levels in biological samples involving tissues and organisms.

In some embodiments, the liposome-complexes are configured to work in vivo in one or more of the following ways:
(a) MOFs when exposed to specific molecules or ions in the biological system, can undergo ion exchange processes. When the MOF comes into contact with metal ions in the solution or when it interacts with ions present in the biological fluids, an exchange of metal ions in the MOF may occur. This ion exchange process may result in the modification of MOF's composition, structure and properties resulting in the altered reactivity or targeted release of encapsulated substances; and/or,
(b) Metal complexes can undergo ligand exchange reactions in the presence of specific biomolecules or ligands present in the biological environment which may result in the activation or alteration of the complex's properties.

Without wishing to be bound by any particular theory, it is generally believed that the complexes disclosed herein detect oxygen via the following mechanism: upon absorption of light, the electron in Cu+ in the MOF makes a transition from the ground singlet state (S0) to a short-lived excited singlet state (Si). The electron then from the singlet excited state (1PS*) either can fluorescence back to the ground state or undergo intersystem crossing (ISC) into an electronically excited triplet state (T1). The excited triplet (3PS*) can undergo two types of reactions. Either it can participate in an electron transfer process with a substrate to form radicals and radical ions which after interaction with oxygen can produce oxygenated products, and/or it can undergo a photochemical process by transferring energy to the surrounding molecular oxygen which results in the conversion of stable triplet oxygen (302) to the short-lived but highly reactive singlet oxygen (102). When the oxygen molecule is added as a quencher, being in the vicinity of the excited species, it can easily interact with the excited state and influence its decay process. This interaction results in quenching i.e., decrease in the emission intensity.

### EXAMPLES

### 1. General Experiments

All the experiments in this example were carried out inside the glovebox under inert conditions, except in any case was demonstrated. Some of the reagents were commercially available and used directly without further purification. ¹H NMR spectra were recorded on a Bruker NMR 500 DRX spectrometer at 500 MHz and referenced to the proton resonance resulting from deuterated chloroform (δ 7.26). Room temperature powder X-ray diffraction data were collected on a Bruker Advance diffractometer using Ni-filtered Cu Kα radiation (λ=1.5406 Å). Data were collected with a step size of 0.05° and at count time of is per step over the range 4°<20<70°. For the product analysis of the liquid phase using GC-MS, the following chromatographic conditions were employed; carrier gas: He, flow rate: 1 mL min⁻¹, injection volume: 5.0 μL, column oven temperature was initially 80° C. and then increased up to 230° C. with the rate of 5° C. per minute, and detector temperature was 250° C. High-resolution mass spectra (HRMS) were recorded on a Q-TOF Bruker instrument, using electrospray ionization (ESI) as the ionization method. UV-vis absorption studies were carried out with the help of Agilent Cary 60 UV-Vis Spectrophotometer in the range of 200-800 nm. For Fit Fluorescence Intensity, Time Resolved and Steady State Photoluminescence (TRPL) studies, QM-8450-22-C System #3733 of HORIBA Scientific was used. The same was used for calculating the quantum yield measurements using the integrating sphere mechanism.

### 2. Synthesis of Metal Complexes

### 2.1 Synthesis of M1, [CuI(CH3CN)4][PF6]

The following synthesis has been done according to the previously reported procedure.¹ Copper Sulphate Pentahydrate (0.5 g, 0.002 mmol) was dissolved in water (11 mL). Potassium hexafluorophosphate (1.5 g, 0.008 mmol) was added to the above solution followed by addition of acetonitrile (1.725 mL). The solution was sonicated well to get a completely soluble mixture. The above solution was transferred to plastic vial to which copper wire scratched with sandpaper was added. The mixture was sonicated well and shaken several times. The solution was then heated in boiling water on heating mantle for 10 minutes. It was then cooled in ice water. The same procedure was repeated for 4-5 times till blue colour of Cu²⁺ disappeared. The reaction mixture was then cooled to room temperature. The copper wire was removed from the vial. The obtained white suspension was cooled to complete the precipitation of the product. The suspension was then washed and centrifuged initially with acetonitrile (0.1 g i.e 0.127 mL) in water (5 mL) followed by acetonitrile (0.2 g i.e. 0.25 mL) in a mixture of ethyl acetate (5 mL) and ethanol (5 mL) and lastly with acetonitrile (0.1 g i.e. 0.127 mL) in ethyl acetate (5 mL). The obtained product was stored in the glovebox.

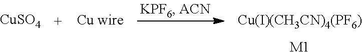

### 2.2 Synthesis of M2, [CuI(bpy)2][BF4]

The following synthesis has been done according to the previously reported procedure.² Tetrakis(acetonitrile)copper(I)tetrafluoroborate (60 mg, 0.190 mmol) was taken in 1 mL dry degassed methanol in glovebox. Bipyridine (60 mg, 0.381 mmol) was dissolved in methanol (5 mL) and then both the solutions were mixed in a round bottom flask containing magnetic bead. The solution was kept for stirring for 3-4 h in the glovebox in N₂ atmosphere till the solvent got evaporated from the flask and red-brown coloured precipitate was obtained. This was followed by washing the precipitate with heptane solvent. After some time, heptane solvent was decanted and final washing was done with dimethyl ether. The obtained product was stored in the glovebox.

### 2.3 Synthesis of A, Tris(2-pyridyl)methane

The following synthesis has been done according to the previously reported procedure.³ 2-methylpyridine (200 mg, 2.14 mmol) and tetrahydrofuran (4.13 mL) were taken in a beaker and cooled to −78° C. in julabo setup. A solution of n-BuLi (1.07 mL) was slowly added to the stirred solution of the above with the help of syringe within 5-7 min. The resulting deep red solution was stirred at −78° C. for 30 min. The reaction temperature was then raised slowly to −20° C. 2-fluoropyridine (104 mg. 1.07 mmol) was added dropwise to the mixture. The reaction temperature was raised to ambient within 20 min. The mixture was refluxed and stirring was done for 1 h in N2 atmosphere at 150° C. The reaction was then cooled to room temperature. 2-fluoropyridine (104 mg, 1.07 mmol) was again added dropwise to the mixture and then finally it was refluxed for 48 h. The solution was then cooled down to room temperature. The mixture was quenched with water and then the organic and aqueous layers were separated. With the help of separating funnel, the aqueous layer was extracted with ethyl acetate. The organic layer was dried over MgSO₄ and the solution was left undisturbed for some time. The obtained yellow solution was then evaporated using rota. Column chromatography of the solution was carried out in pure ethyl acetate mixture. The formed product from the column was then evaporated using rota. The mixture was then layered with diethyl ether. The yellow crystalline precipitate was separated and the product was collected by filtration. The product was then finally washed with diethyl ether.

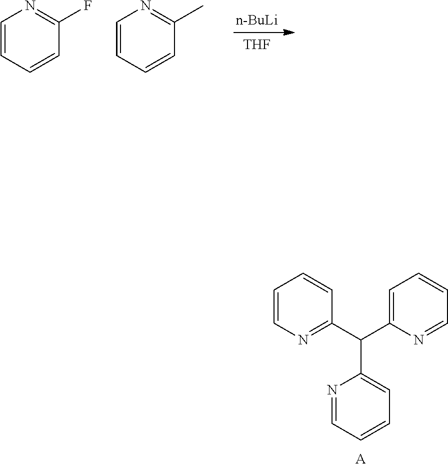

### 2.4 Synthesis of B, Tri(o-tolyl)phosphine

The following synthesis has been done according to the previously reported procedure⁴ A magnetic stirrer was placed. A schlenk flask was connected via N₂ line to which magnesium turnings (0.75 g, 31.13 mmol) were added. A pinch of iodine was added to it and then the schlenk flask was heated with hot gun. Once the flask was hot, the magnesium turnings were allowed to rotate for 15-20 min. A gradual change in the colour was observed from colourless to wine to brown. 2-Bromotoluene was added to the flask containing the magnesium turnings. Dry tetrahydrofuran (20-25 mL) was taken in another schlenk flask. THF solution was transferred from the schlenk flask to the above mixture solution dropwise with the help of cannula under stirring at 80° C. The solution was then refluxed for 45 min and stirring was done at 100° C. The setup was allowed to cool back to room temperature. Nitrogen was flushed thrice into the setup and then slowly phosphorous trichloride was purged with syringe back into the setup. The reaction was then stirred for 24 h. The reaction was allowed to cool and then quenched with water. The aqueous phase was degassed with diethyl ether solution and the combined organic phases were dried over MgSO₄. The solution was then filtered and then the solvent was evaporated with rota. The obtained product was stored in glovebox.

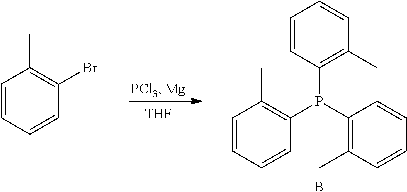

### 2.5 Synthesis of X1, Cu(I) Complex

The following synthesis has been done according to the previously reported procedure.⁵ Tetrakis(acetonitrile)copper(I)hexafluorophosphate (45.09 mg, 0.121 mmol) was dissolved in degassed dichloromethane (6 mL) in a round bottom flask and tris(2-pyridyl)methane (30 mg, 0.121 mmol) was stoichiometrically added to the above solution. The reaction mixture was kept for stirring for 5-10 min inside the glovebox. Tri(o-tolyl)phosphine (38.65 mg. 0.127 mmol) was added to the above round bottom flask and the reaction mixture was stirred for 12 h at room temperature. After 12 h, the round bottom flask was taken out of the glove box and the solvent was evaporated using rota. The product was washed with diethyl ether and then with pentane. The obtained product was stored in glovebox.

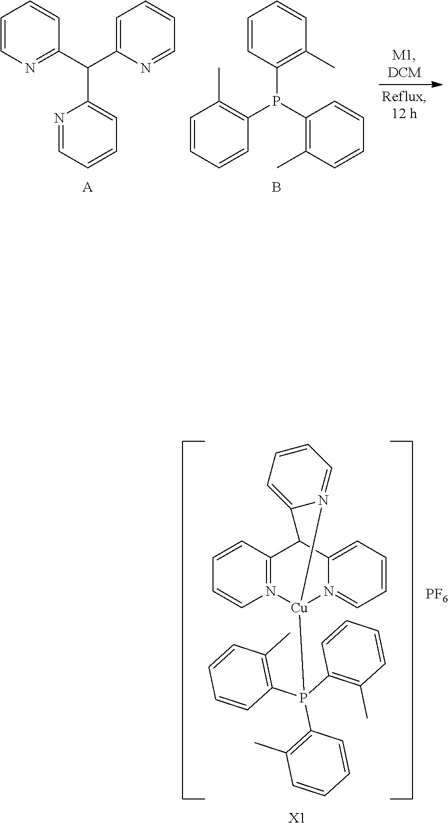

### 2.6 Synthesis of X2, Cu(I) Complex

Tetrakis(acetonitrile)copper(I)hexafluorophosphate (30 mg, 0.080 mmol) was dissolved in degassed dichloromethane (4 mL) in a round bottom flask and tris(2-pyridyl)methane (19.78 mg, 0.080 mmol) was stoichiometrically added to the above solution. The reaction mixture was kept for stirring for 5-10 min inside the glovebox. Methyldiphenylphosphine (16.81 mg, 0.084 mmol) was added to the above round bottom flask and the reaction mixture was stirred for 12 h at room temperature. After 12 h, the round bottom flask was taken out of the glove box and the solvent was evaporated using rota. The product was washed with diethyl ether and then with pentane. The obtained product was stored in glovebox.

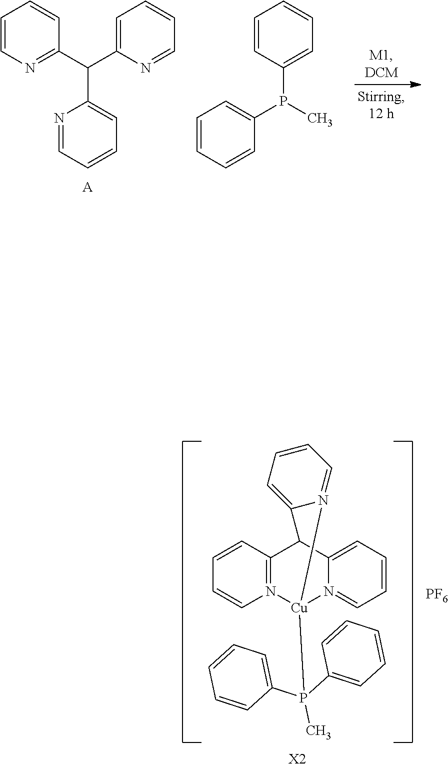

### 2.7 Synthesis of X3, Cu(I) Complex

Tetrakis(acetonitrile)copper(I)hexafluorophosphate (30 mg, 0.080 mmol) was dissolved in degassed dichloromethane (4 mL) in a round bottom flask and tris(2-pyridyl)methane (19.78 mg, 0.080 mmol) was stoichiometrically added to the above solution. The reaction mixture was kept for stirring for 5-10 min inside the glovebox. Tricyclohexylphosphine (23.55 mg, 0.084 mmol) was added to the above round bottom flask and the reaction mixture was stirred for 12 h at room temperature. After 12 h, the round bottom flask was taken out of the glove box and the solvent was evaporated using rota. The product was washed with diethyl ether and then with pentane. The obtained product was stored in glovebox.

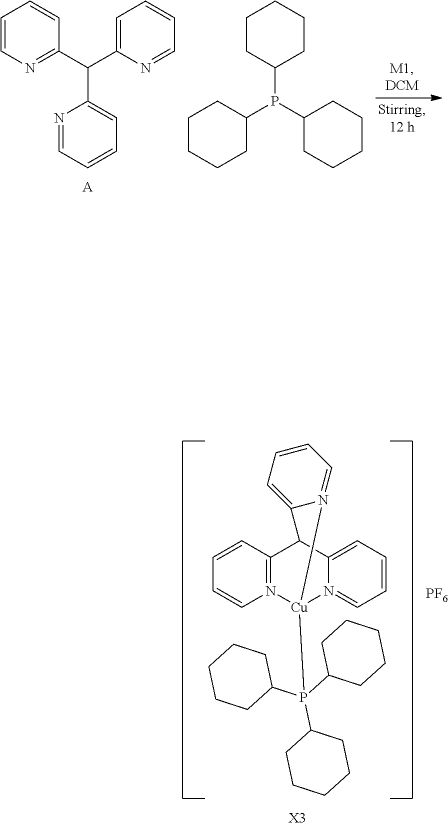

### 2.8 Synthesis of X4, Cu(I) Complex

Tetrakis(acetonitrile)copper(I)hexafluorophosphate (30 mg, 0.080 mmol) was dissolved in degassed dichloromethane (4 mL) in a round bottom flask and tris(2-pyridyl)methane (19.78 mg, 0.080 mmol) was stoichiometrically added to the above solution. The reaction mixture was kept for stirring for 5-10 min inside the glovebox. Phosphorous trichloride (23.55 mg, 0.084 mmol) was added to the above round bottom flask and the reaction mixture was stirred for 12 h at room temperature. After 12 h, the round bottom flask was taken out of the glove box and the solvent was evaporated using rota. The product was washed with diethyl ether and then with pentane. The obtained product was stored in glovebox.

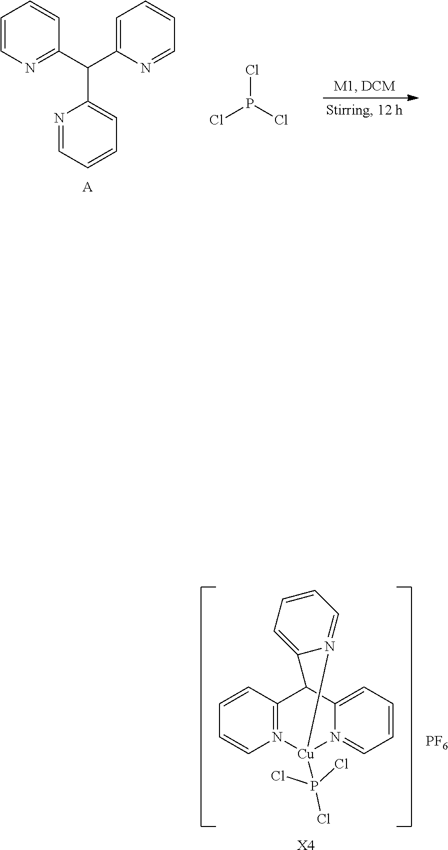

### 2.9 Synthesis of X5, Cu(I) Complex

Tetrakis(acetonitrile)copper(I)hexafluorophosphate (30 mg, 0.080 mmol) was dissolved in degassed dichloromethane (4 mL) in a round bottom flask and tris(2-pyridyl)methane (19.78 mg, 0,080 mmol) was stoichiometrically added to the above solution. The reaction mixture was kept for stirring for 5-10 min inside the glovebox. Triphenylphosphite (26.06 mg, 0.084 mmol) was added to the above round bottom flask and the reaction mixture was stirred for 12 h at room temperature. After 12 h, the round bottom flask was taken out of the glove box and the solvent was evaporated using rota. The product was washed with diethyl ether and then with pentane. The obtained product was stored in glovebox.

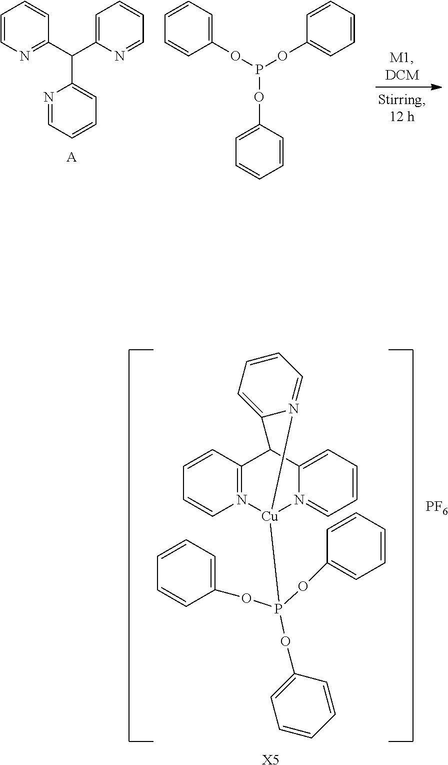

### 3. Synthesis

### 3.1 Synthesis of X6, Cu(I) Complex

The below complex was synthesised whose structure was analogous to the reported one.^{6 7} Tetrakis(acetonitrile)copper(I)hexafluorophosphate (80 mg, 0.214 mmol) was dissolved in degassed dichloromethane (3 mL) in a round bottom flask. 1,2-bis(diphenylphosphino) benzene (191.08 mg. 0.428 mmol) was added to the above round bottom flask and the reaction mixture was stirred for 24 h at room temperature. After 24 h, the round bottom flask was taken out of the glove box and the solvent was evaporated using rota. The product was washed with diethyl ether and then with pentane. The obtained product was stored in glovebox.

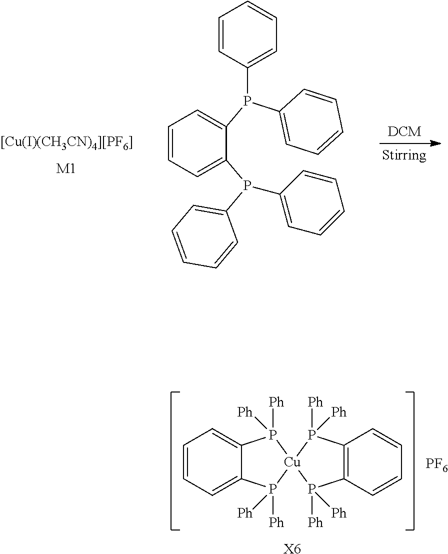

### 3.2 Synthesis of D, 2-(2,4-Difluorophenylpyridine)

The following synthesis has been done according to the previously reported procedure.⁸ 1M sodium bicarbonate solution was prepared by dissolving 0.53 g of sodium bicarbonate in 5 mL water. It was then degassed using nitrogen gas via schlenk line. 2,4-Difluorophenylboronic acid (100 mg, 0.632 mmol) was taken in a schlenk round bottom flask inside the glove box. Tetrakis(triphenylphosphine)palladium(0) (22 mg, 0.019 mmol) was dissolved in 1.5 mL of tetrahydrofuran and the solution was added to the above flask containing 2,4-Difluorophenylboronic acid solution. The flask was then taken out of the glovebox. A magnetic bead was added to the above flask. With the help of syringe, 2-Bromopyridine (100 mg, 0.632 mmol) was added to the flask followed by addition of degassed Na₂CO₃ (900 μL) to the flask via nitrogen line. The above solution was then placed on an oil bath at 145° C. -150° C. and the reaction was allowed to reflux for 24 h. TLC of the formed mixture was checked in dichloromethane in short UV range. The cooled crude mixture was poured onto water in a separating funnel and extracted with DCM three times. The lower layer containing the product dissolved in DCM was collected in a beaker leaving the top layer in the funnel. This procedure was repeated several times. The mixture was then dried over anhydrous magnesium sulphate. The beaker containing the solution was left undisturbed for some time. The mixture was then collected in round bottom flask and the solvent was then evaporated using rota to get the product. Finally, silica column purification was done for the product in 25% ethyl acetate in hexane mixture which gave a yellow liquid as the final product.

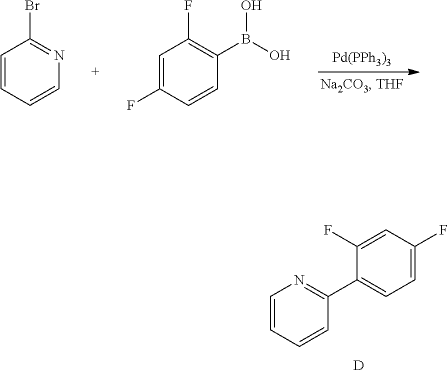

### 3.3 Synthesis of X7, Metalation of the Ir Complex with 2-(2,4-Difluorophenylpyridine) Ligand

The following synthesis has been done according to the previously reported procedure⁹ Solution of 2-methoxyethanol in water (2:1, 1 ml) was first degassed in a schlenk flask. 2(2,4-Difluorophenylpyridine) ligand (27.24 mg, 0.1425 mmol) and iridium trichloride (20 mg, 0.057 mmol) were added to the above schlenk flask. The mixture was refluxed and stirring was done for 12-18 h in N₂ atmosphere at 150° C. The reaction mixture was allowed to cool down for some time and then it was quenched with water. The product was then washed and filtered with diethyl ether followed by ethanol. Yellow solid was obtained as the final product.

### 3.4 Synthesis of X8, Tris-Cyclometalated Ir(III) Complex

The following synthesis has been done according to the previously reported procedure.¹⁰ The above synthesised Ir ligand (60 mg, 0.049 mmol) was taken in a schlenk flask under nitrogen supply. Solution of glycerol (2 mL) was degassed in a schlenk flask and then transferred to the above flask followed by the addition of 2-(2,4-Difluorophenylpyridine) ligand (120 mg, 0.627 mmol) to it. Potassium carbonate (100 mg, 0.723 mmol) was added to the schlenk flask and a magnetic bead was added to it. The reaction mixture was then kept for stirring and was refluxed for 18 h. The reaction was allowed to cool and was quenched with water (4 mL). The product was washed and filtered with methanol, diethyl ether and lastly with hexane. The residue was kept in oven for drying at 60° C. The filtrate was then evaporated using rota. Black-brown product was obtained.

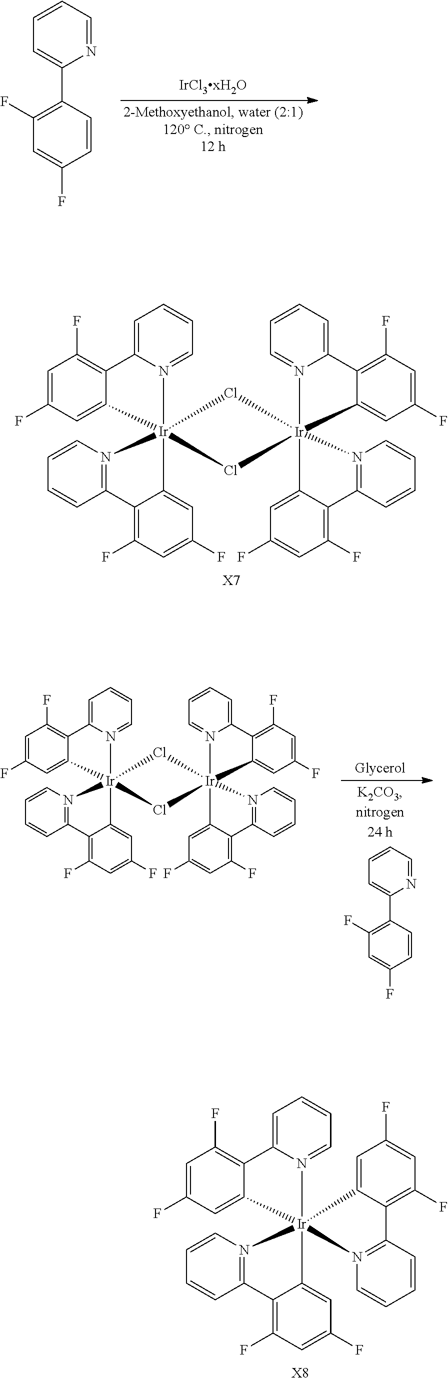

### 4. Synthesis of MOFs

### 4.1 Synthesis of X9, Copper-Functionalized Mono-Phosphine-MOF

The following synthesis has been done according to the previously reported procedure.¹¹ The mono-phosphine-MOF was prepared from 4′,4′″,4′″″-phosphanetriyltris([1,1′-biphenyl]-4-carboxylate i.e. [P(ArCO₂)₃] as a bridging linker and Zr-oxo-hydroxo SBUs. Benzoic acid was used as a modulator in DMF. Firstly, benzoic acid (50 mg, 0.409 mmol) and the bridging linker i.e. [P(ArCO₂)₃](7.8 mg, 0.015 mmol) were weighed in two separate vials and then taken in the glove box where ZrCl₄ (5.8 mg, 0.024 mmol) was weighed separately. Inside the glovebox, degassed DMF (0.5 mL) was added to the vial containing the linker and then mixed with benzoic acid. This was followed by the addition of degassed DMF (0.5 mL) to ZrCl₄. The above solutions were then dissolved properly in a single glass vial. The mixture solution was then added to the glass tube. The tube was then sealed and placed in a preheated oven at 90° C. for 5 days. After 5 days, the tube was taken out of the oven and carefully taken inside the glovebox where it was broken to get the white crystalline solid, MOF-P. The MOF-P was then taken in the centrifuge tube and washed with DMF thrice. The copper-functionalized mono-phosphine MOF, X9 was synthesised by charging MOF-P in THF into a vial followed by addition of solution of CuI in THF at room temperature. The resultant solid was centrifuged out of suspension and washed with THF 4-5 times.

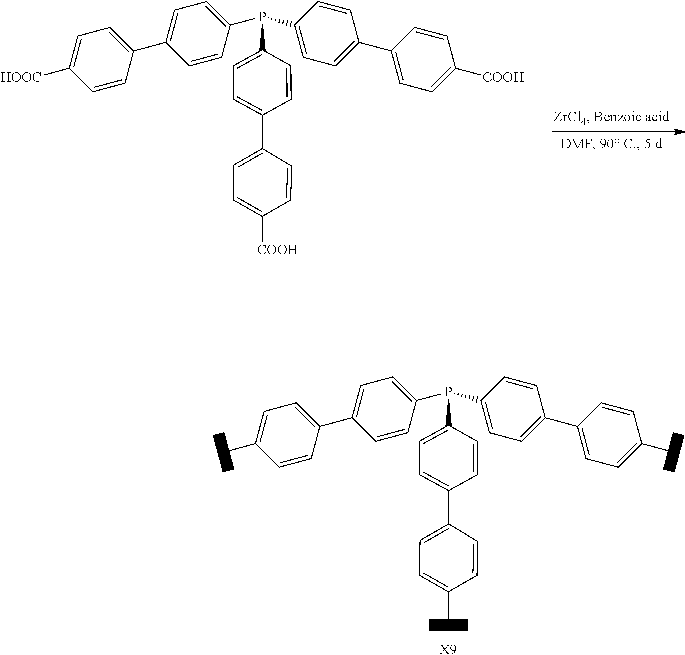

### 4.2 Synthesis of E, dimethyl-2′-amino-[1,1′:4,1″-terephenyl]-4,4″-dicarboxylate

The following synthesis has been done according to the previously reported procedure.^{12 13} To a mixed solution of water (10 mL) and deoxygenated N,N-dimethylformamide (10 mL) under N₂ atmosphere, 2,5-dibromoaniline (200 mg, 0.790 mmol), 4-(methoxycarboxyl)phenylboronic acid (570 mg, 3.180 mmol) and Pd(OAc)₂ (1.5 mg, 0.0067 mmol) were added in a 50 mL schlenk flask and refluxed at 105° C. for 16 h. The reaction mixture was then cooled to room temperature and a greenish-yellow precipitate was obtained which was collected via centrifugation and then washed with water. With the help of a mixture solution of CH₂Cl₂:EtOAc (50:0.5 by volume), the crude product was purified by silica gel column chromatography.

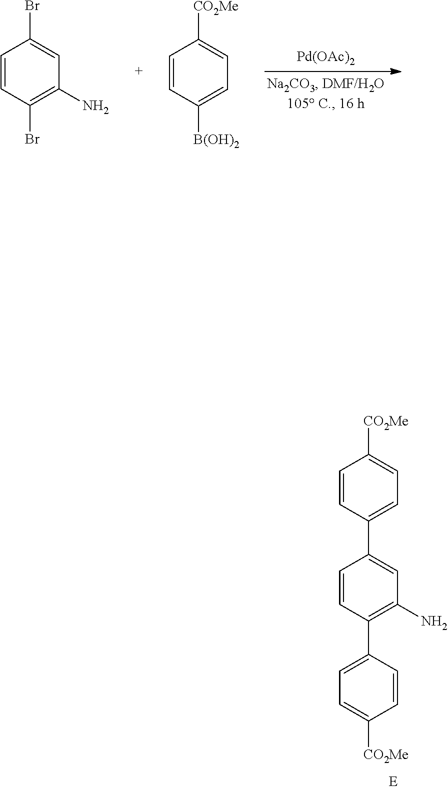 [SMILES: Nc1cc(Br)ccc1Br ; COC(=O)c1ccc(B(O)O)cc1 ; COC(=O)c1ccc(-c2ccc(-c3ccc(C(=O)OC)cc3)c(N)c2)cc1 ; "Pd(OAc)2"]

### 4.3 Synthesis of H2TPDC-NH2, 2′-Amino-[1,1′:4′,1″-terphenyl]-4,4″-dicarboxylic acid

The following synthesis has been done according to the previously reported procedure.¹³ A solution of KOH in methanol (5.50M, 20 mL) was prepared and then added to the flask containing the suspension of E (200 mg, 0.553 mmol) in THF (7-8 mL). The solution mixture was refluxed overnight at 70° C. Next day, the reaction mixture was cooled to room temperature and the precipitate was collected through centrifugation. The colourless solid was then suspended to THF (10 mL) and trifluoroacetic acid (TFA) was added dropwise to the above until a pH 2-3 was obtained. The mixture was then stirred for 1 h at room temperature followed by addition of plenty of water to obtain immediate precipitation of light-yellow solid as the product.

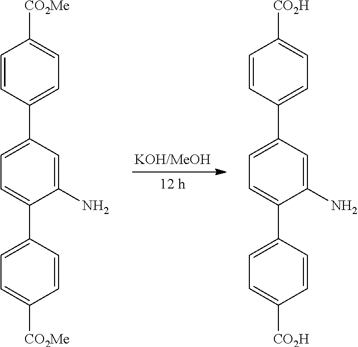 [SMILES: COC(=O)c1ccc(-c2ccc(-c3ccc(C(=O)OC)cc3)c(N)c2)cc1 ; Nc1cc(-c2ccc(C(=O)O)cc2)ccc1-c1ccc(C(=O)O)cc1]

### 4.4 Synthesis of X10, UiO-68-NH2 MOF

The following synthesis has been done according to the previously reported procedure.^{13,14} Benzoic acid (73.0 mg, 0.6 mmol) and H₂TPDC-NH₂ (10.0 mg, 0.03 mmol) were dissolved in DMF solution (1.2 mL). To the above, ZrCl₄ (7.0 mg, 0.03 mmol) was added and the resulting mixture solution was sonicated for a few minutes. It was then kept in a preheated oven at 70° C. for 3 d. The mixture solution was then cooled to room temperature and the crystalline solid was isolated by centrifugation. It was then washed with DMF several times to get X10.

### 4.5 Synthesis of X11, Pyrim-MOF-Cu

The following synthesis has been done according to the previously reported procedure.¹⁴ Inside the glovebox, the synthesised UiO-68-NH₂ MOF (X10) was taken in deoxygenated DMF (1 mL) in a centrifuge tube. To this, 2-pyridinecarboxaldehyde (20 μL, 0.210 mmol) was added. The resultant mixture was left overnight. The mixture was then vortexed several times followed by multiple washing of MOF with DMF to get pyrim-UiO MOF as light brown solid.

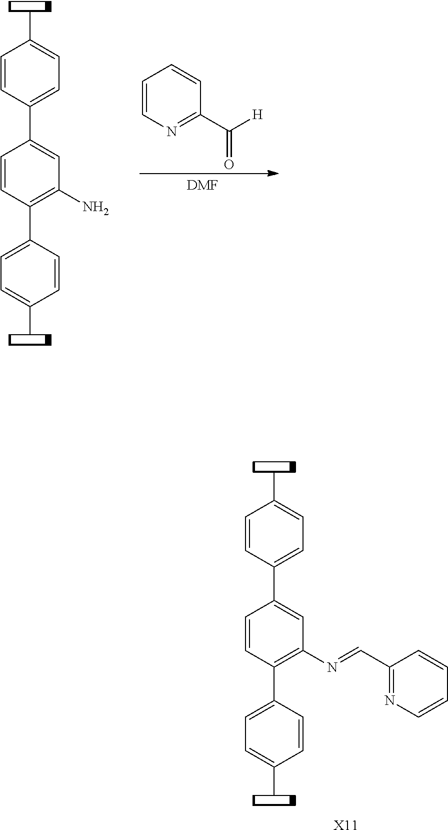

### 4.6 Synthesis of X12, Pyrim-UiO-CuI

A robust copper-pyridylimine-functionalized MOF i.e. pyrim-MOF-CuI (X12) was prepared by charging pyrim-UiO MOF (X11) in THF into a vial followed by addition of solution of CuI in THF at room temperature. The resultant white coloured solid was centrifuged out of suspension and washed with THF 4-5 times.

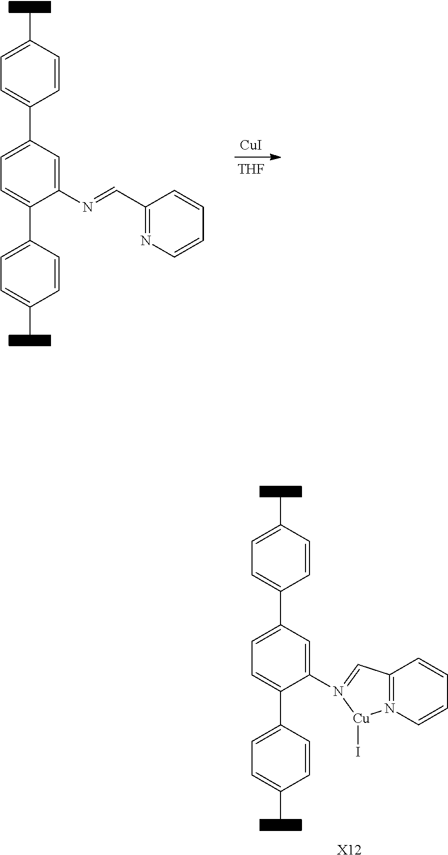

### 5. Phosphorescence Studies

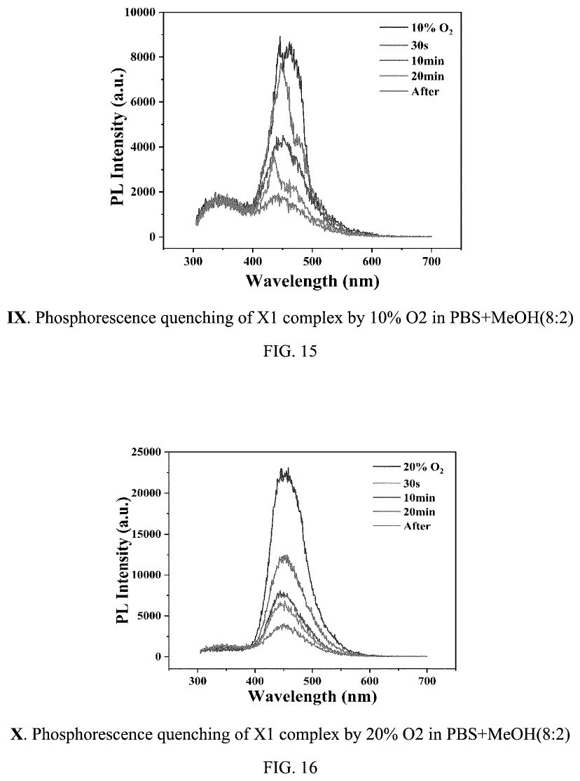

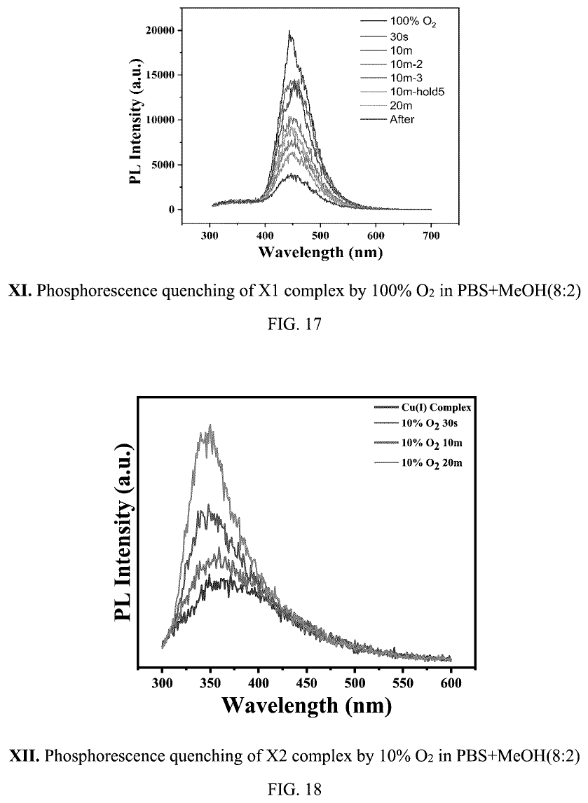

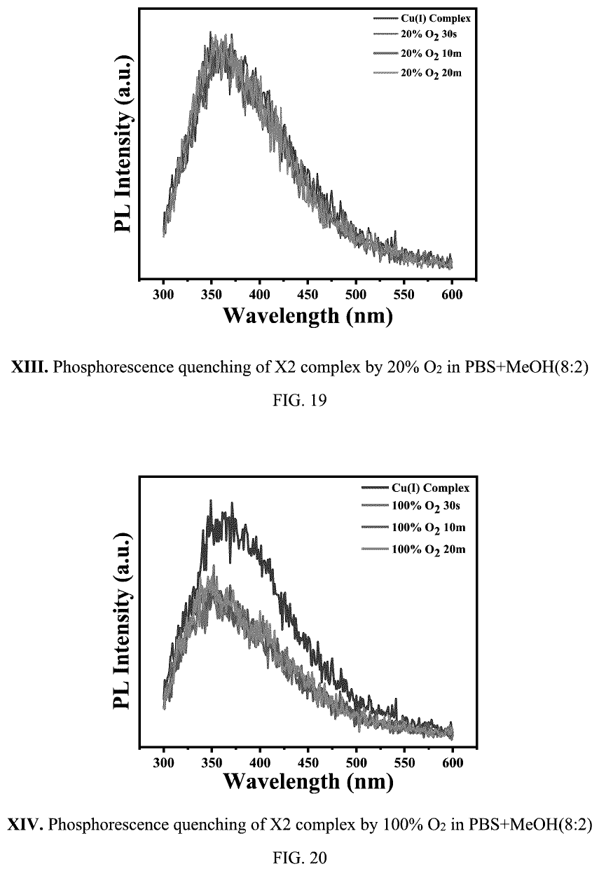

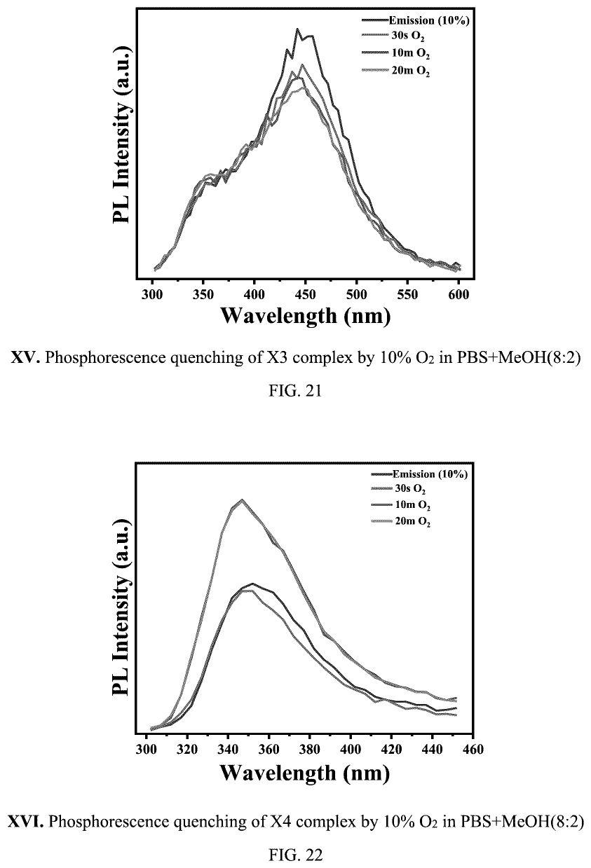

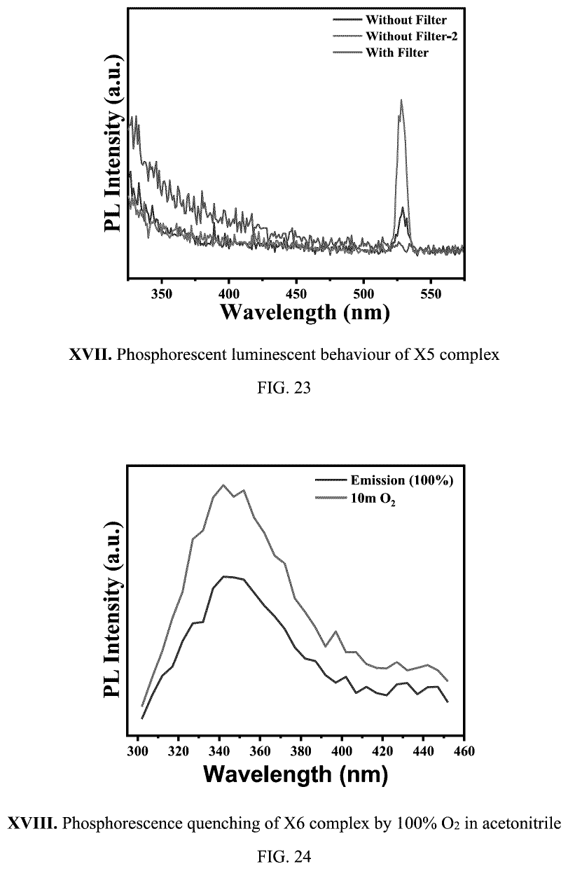

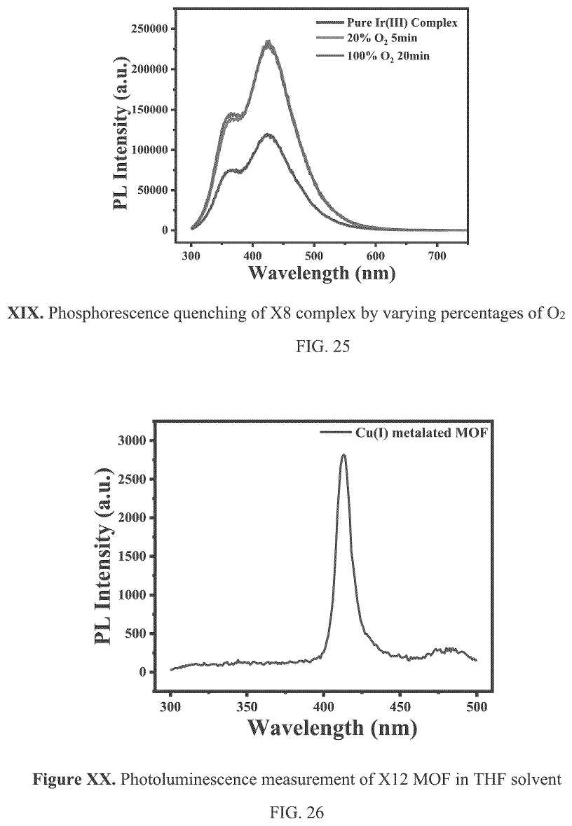

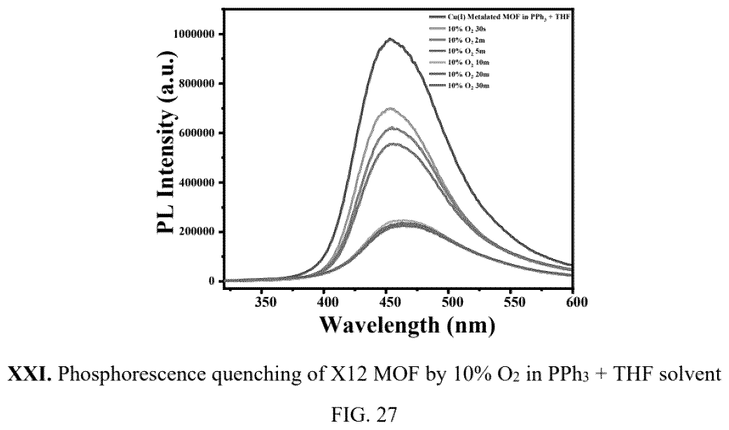

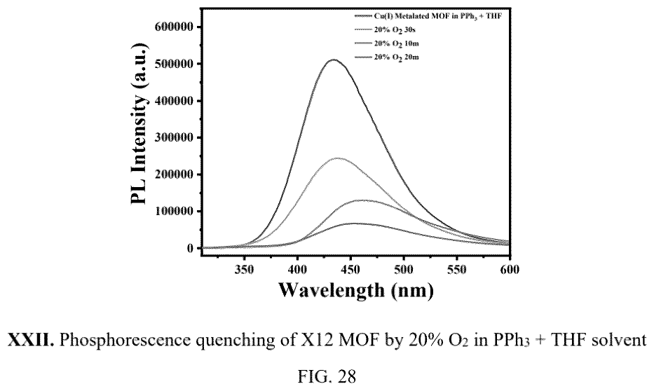

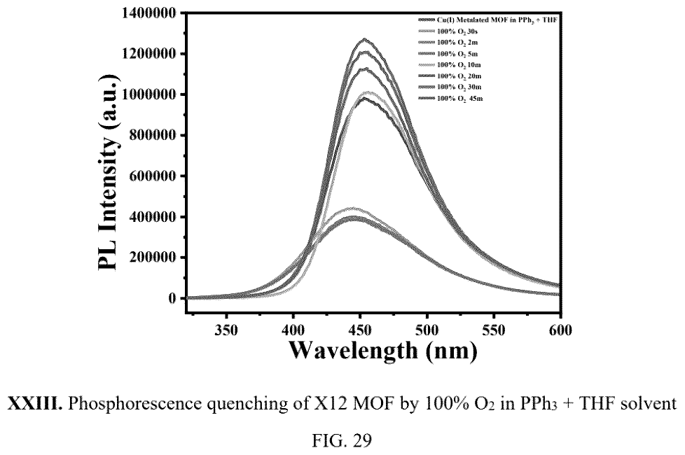

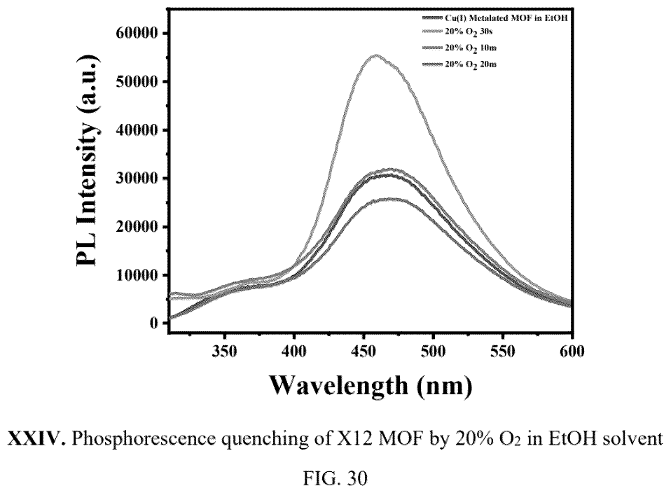

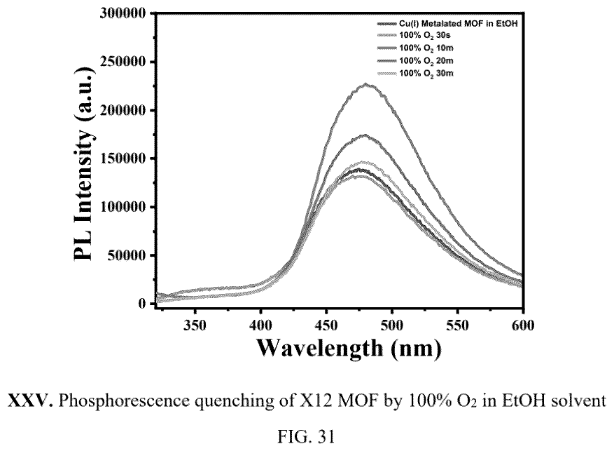

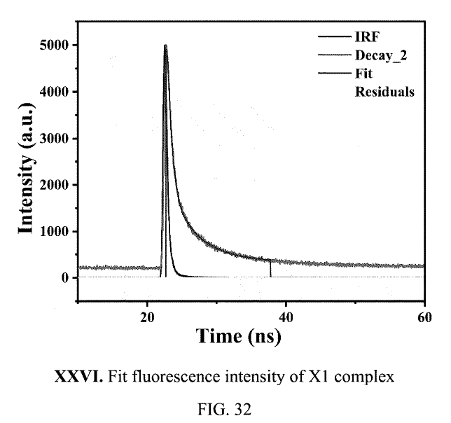

Time Resolved and Steady State Photoluminescence Studies (TRPL) were carried out to study the photoluminescence behaviour of both the complexes and the MOF (FIGS. 14-32). Initially, the emission wavelength of the desired complex/MOF was determined using the excitation wavelength values obtained from UV studies. Then, in order to study the phosphorescence quenching, sample solutions of complexes and thin films of MOF were prepared in different solvents. Balloons were filled with different concentrations of oxygen and nitrogen gases with the help of a glass T which were then bubbled into the sample solutions of complexes in glass cuvettes and films of MOF prepared on the glass slide. By varying the percentages of oxygen and nitrogen gases, the phosphorescence quenching behaviour was studied. The below Tables provide the percent O2 quenched as a function of time as a function of oxygen concentration for exemplary complexes as contemplated herein.

TABLE I
Phosphorescence quenching (%) of X1
complex by 10% O2 in PBS + MeOH(8:2)

| | |
|---|---|
| Time | % O₂ Quenching |

| | | |
|---|---|---|
| 30 | s | 17.37% |
| 10 | min | 42.00% |
| 20 | min | 61.00% |
| 1 | h | 75.00% |

TABLE II
Phosphorescence quenching (%) of X1
complex by 20% O2 in PBS + MeOH(8:2)

| | |
|---|---|
| Time | % O₂ Quenching |

| | | |
|---|---|---|
| 30 | s | 45.00% |
| 10 | min | 66.00% |
| 20 | min | 71.00% |
| 1 | h | 82.00% |

TABLE III
Phosphorescence quenching (%) of X1
complex by 100% O2 in PBS + MeOH(8:2

| | |
|---|---|
| Time | % O₂ Quenching |

| | | |
|---|---|---|
| 30 | s | 14.00% |
| 10 | min | 40.00% |
| 20 | min | 48.00% |
| 1 | h | 74.00% |

TABLE IV
Phosphorescence quenching (%) of X8
complex by varying percentages of O2

| Time | % O₂ | % O₂ quenching |
|---|---|---|
| 5 min | 20% | 10.55% |
| 20 min | 100% | 11.15% |

TABLE V
Phosphorescence quenching (%) of X12
MOF by 10% O2 in PPh3 + THF solvent

| | |
|---|---|
| Time | % O₂ Quenching |

| | | |
|---|---|---|
| 30 | s | 33.00% |
| 2 | min | 39.00% |
| 5 | min | 44.00% |
| 10 | min | 72.52% |
| 20 | min | 73.43% |
| 30 | min | 74.14% |

TABLE VI
Phosphorescence quenching (%) of X12
MOF by 20% O2 in PPh3 + THF solvent

| | |
|---|---|
| Time | % O₂ Quenching |

| | | |
|---|---|---|
| 30 | s | 49.00% |
| 10 | min | 74.00% |
| 20 | min | 85.00% |

### 6. Quantum Yield Measurements

The quantum yield measurements are done using the integrating sphere method.

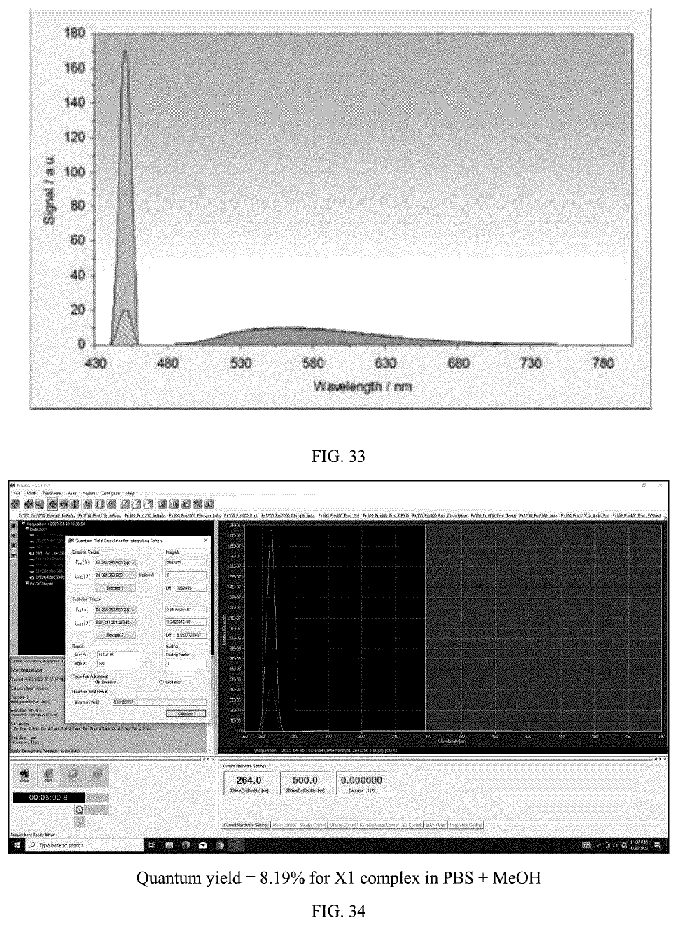

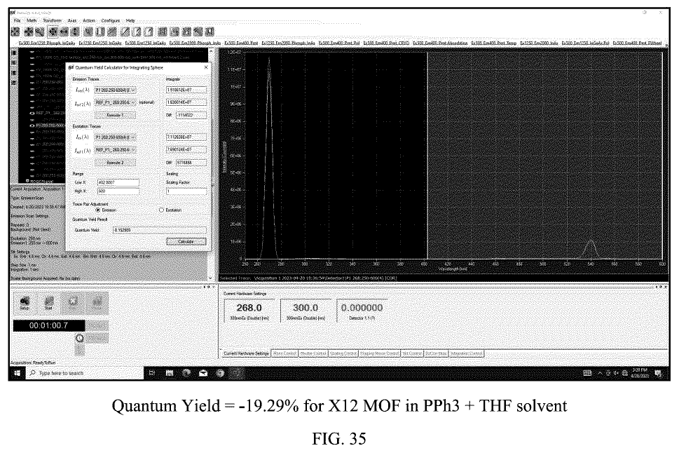

Two spectral scans are illustrated graphically in FIG. 33 by scanning the emission monochromator while maintaining a fixed excitation monochromator at 450 nm. The reference scatterer in the first scan (blue) should have a diffuse reflectance of 100%. The sample under investigation is then scanned in the second scan (red). This sample displays emission (red, solid) and reflection (red, hatched). Here, the region of the blue graph (starting from the base line) is referred to as E_{ref}, the region of the red hatching area as Eₛₐₘ and the region of the red solid area as Lₛₐₘ.

With the help of the above mechanism, it is possible to calculate the below mentioned quantities:

| | |
|---|---|
| Reflectance at λ_{exc} | $R ( {λ}_{exc} ) = \frac{{E}_{Sam}}{{E}_{Ref}}$ |
| Absorbance at λ_{exc} | $A ( {λ}_{exc} ) = {log}_{10} ( \frac{{E}_{Ref}}{{E}_{Sam}} )$ |
| Quantum Yield | $η = \frac{{L}_{Sam}}{{E}_{Ref} - {E}_{Sam}}$ |

In a preferred embodiment the complex has a quantum yield of between 8% and 10%. The quantum yield value of the synthesised [Cu(tpym)(P(o-tol)₃)]PF₆ complex is reported in DCM solvent.

Quantum yield=8.19% for X1 complex in PBS+MeOH

Quantum Yield=−19.29% for X12 MOF in PPh₃+THF solvent

### EQUIVALENTS AND SCOPE

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A complex of Formula (I): 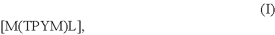
or a salt thereof, wherein:
M is Cu(I), Ir, Rh, Ag, Co, Fe, Ru, Ni, Zn, or Au;
TPYM is Tris(2-pyridyl)methane, and
L is a monophosphine ligand.

2. The complex of claim 1, wherein the monophosphine ligand comprises a structure: 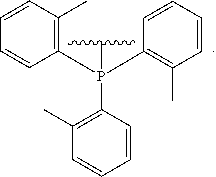
or a salt thereof, and wherein [symbol] indicates a point of attachment.

3. The complex of claim 1, wherein the monophosphine ligand comprises a structure: 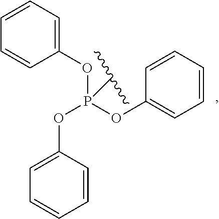
or a salt thereof, and wherein [symbol] indicates a point of attachment.

4. The complex of claim 1, wherein the monophosphine ligand comprises a structure: 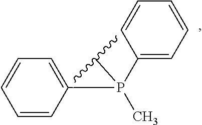
or a salt thereof, and wherein [symbol] indicates a point of attachment.

5. The complex of claim 1, wherein the monophosphine ligand comprises a structure 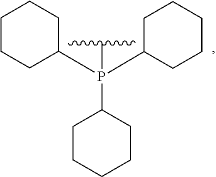
or a salt thereof, and wherein [symbol] indicates a point of attachment.

6. The complex of claim 1, wherein the monophosphine ligand comprises a structure: 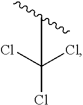 [SMILES: *C(Cl)(Cl)Cl]
or a salt thereof, and wherein [symbol] indicates a point of attachment.

7. A metal organic framework composition comprising:
a plurality of metal clusters, wherein at least one metal cluster comprises a metal ion; and
a plurality of ligands coordinating with the plurality of metal clusters, wherein the plurality of ligands comprises a triphenyldicarboxylic acid (TPDC) group or a triphenylphosphine group.

8. The metal organic framework of claim 7, wherein the plurality of ligands comprises a structure: 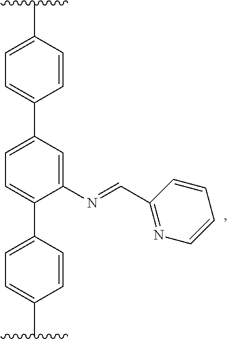 [SMILES: *c1ccc(-c2ccc(-c3ccc(*)cc3)c(/N=C/c3ccccn3)c2)cc1]
or a salt thereof, and wherein [symbol] indicates a point of attachment.

9. The metal organic framework of claim 7, wherein plurality of ligands comprises a structure: 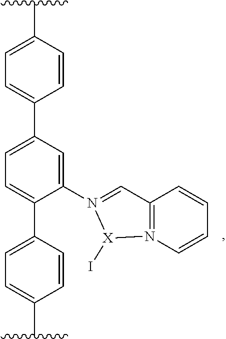
or a salt thereof, wherein X is Cu, Co, Ag, or Ir, and wherein [symbol] indicates a point of attachment.

10. The metal organic framework of claim 7, wherein the plurality of ligands comprises a structure: 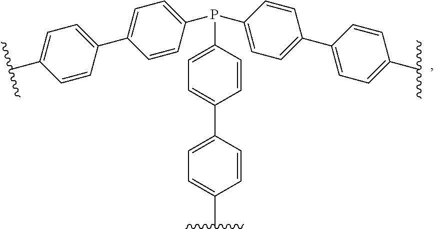 [SMILES: *c1ccc(-c2ccc(P(c3ccc(-c4ccc(*)cc4)cc3)c3ccc(-c4ccc(*)cc4)cc3)cc2)cc1]
or a salt thereof, and wherein [symbol] indicates a point of attachment.

11. The metal organic framework of claim 7, wherein the plurality of metal cluster comprises UiO-68 (Zr₆O₄(OH)₄).

12. The metal organic framework of claim 7, wherein the metal organic framework has one octahedral cavity and eight tetrahedral cavities.

13. The metal organic framework of claim 7, wherein at least one cargo is encapsulated within the octahedral cavity.

14. The metal organic framework of claim 7, wherein at least one cargo is encapsulated within the tetrahedral cavities.

15. The metal organic framework of claim 7, further comprising one or more cargos conjugated to the metal organic framework.

16. The metal organic framework of claim 7, wherein the solution further comprises triphenylphosphine and THF.

17. The metal organic framework of claim 7, wherein the metal organic framework is a UiO-type metal organic framework.

18. A metal organic framework of claim 7, wherein at least one metal cluster comprises a metal ion.

19. A method of detecting intratumoral oxygen tension in a subject having, or suspected of having, a solid tumor, comprising:
administering one or more of a complex to the subject and a metal organic framework to the subject, wherein the complex comprises a transition metal, a TPYM group, and a monophosphine ligand and wherein the metal organic framework comprises a plurality of metal clusters and a plurality of ligands coordinating with the plurality of metal clusters, wherein at least one metal cluster comprises a metal ion, and wherein the plurality of ligands comprises a triphenyldicarboxylic acid (TPDC) group or triphenylphosphine group;
non-invasively monitoring one or more of a decrease in a photoluminescence intensity of the complex within the solid tumor, relative to the photoluminescence intensity of the complex in blood and a decrease in a photoluminescence intensity of the metal organic framework within the solid tumor, relative to the photoluminescence intensity of the metal organic framework in blood; and
using a difference in photoluminescence intensity to determine the intratumoral oxygen tension in the subject.

20. The method of claim 7, further comprising treating the subject based on a measured oxygen tension with a drug that inhibits a HIF-1 alpha pathway.
